(19) United States
(12) Patent Application Publication
(10) Pub. No.: US 2025/0011258 A1
(43) Pub. Date: Jan. 9, 2025

(54) SYSTEMS AND METHODS FOR HYDROCARBON PROCESSING

(71) Applicant: Enerflex US Holdings Inc., Houston, TX (US)

(72) Inventor: Timothy W. Oneal, Tulsa, OK (US)

(73) Assignee: Enerflex US Holdings Inc., Houston, TX (US)

(21) Appl. No.: 18/775,853

(22) Filed: Jul. 17, 2024

Related U.S. Application Data
(63) Continuation-in-part of application No. 18/539,085, filed on Dec. 13, 2023.
Application No. 18/539,085 is a continuation of application No. 17/705,096, filed on Mar. 25, 2022, now Pat. No. 11,884,621.
(60) Provisional application No. 63/586,924, filed on Sep. 29, 2023.
(60) Provisional application No. 63/166,179, filed on Mar. 25, 2021.

(51) Int. Cl. C07C 7/09; B01D 3/00; B01D 3/14; C07C 7/00; C07C 9/04; C10L 3/10; F25J 1/00

(52) U.S. Cl. CPC C07C 7/09; B01D 3/007; B01D 3/143; C07C 7/005; C07C 9/04; C10L 3/10; F25J 1/0022

(57) ABSTRACT

A process for the separation of a natural gas stream is provided. The process includes receiving a natural gas, cooling and separating a first portion of the gas, and providing the cooled gas reflux streams to a fractionator. The process includes directing another portion of the gas into the fractionator. The fractionator forms a first liquid product stream and a first vapor effluent stream.

### CROSS-REFERENCE TO RELATED APPLICATIONS

This application claims the benefit of U.S. Provisional Patent Application No. 63/586,924 (pending), filed on Sep. 29, 2023, and entitled “Systems and Methods for Hydrocarbon Processing,” the entirety of which is incorporated herein by reference. This application is also a Continuation-in-Part of U.S. patent application Ser. No. 18/539,085 (pending), filed on Dec. 13, 2023, and entitled “System, Apparatus, and Method for Hydrocarbon Processing;” which is Continuation of U.S. Pat. No. 11,884,621 (issued), filed on Mar. 25, 2022, and entitled “System, Apparatus, and Method for Hydrocarbon Processing;” which claims the benefit of U.S. Provisional Patent Application No. 63/166,179 (expired), filed on Mar. 25, 2021, and entitled “System, Apparatus, and Method for Hydrocarbon Processing;” the entireties of each of which are incorporated herein by reference, for all purposes, and made a part of the present disclosure.

### FIELD

This present disclosure relates, generally, to hydrocarbon processing, and, more directly, to systems and methods for the separation of fluids (gas and/or liquid) containing hydrocarbons. More specifically, the disclosed systems and methods are particularly relevant to separation and recovery techniques in natural gas liquid/liquid petroleum gas (NGL/LPG) processing systems. In that respect, the systems and methods disclosed herein are particularly suited or applicable to the separation of ethane, propane, and/or heavier hydrocarbons from such fluid streams.

### BACKGROUND

The separation of natural gas liquid (NGL) from a natural gas stream is typically performed at a centralized processing plant using a process that is similar to processes used to dehydrate natural gas. Common techniques for removing NGL from a natural gas stream include the absorption process and the cryogenic expander process. Often, operators of a natural gas processing plant are compensated based, at least partially, on the capacity of natural gas processed at the plant. Due to equipment limitations, often such plants are limited in the capacity of natural gas that can be processed. It would be desirable to be capable of increasing the capacity of such plants without having to replace the existing architecture of the plant and without overloading the existing equipment of the plant.

### BRIEF SUMMARY

Some embodiments of the present disclosure include a process for separating a natural gas stream. The process includes receiving an input stream of natural gas, separating the input stream into a first input stream and a second input stream, and cooling and separating the first input stream into a first reflux stream and a second reflux stream. The process includes directing the first reflux stream into a first fractionator, and directing the second reflux stream into the first fractionator. The second reflux stream is input into the first fractionator at a location that is below a location where the first reflux stream is input into the first fractionator. The process includes directing the second input stream into the first fractionator. The second input stream is input into the first fractionator at a location that is above the location where the second reflux stream is input into the first fractionator. The process includes operating the first fractionator to fractionate the second input stream, the first reflux stream, and the second reflux stream, thereby forming a first liquid product stream and a first vapor effluent stream in the first fractionator.

Some embodiments of the present disclosure include a process for separating a natural gas stream. The process includes receiving an input stream of natural gas, separating the input stream into a first input stream and a second input stream, and cooling and separating the first input stream into a first reflux stream and a second reflux stream. The process includes directing the first reflux stream into a first fractionator, directing the second reflux stream into the first fractionator at a location that is below a location where the first reflux stream is input into the first fractionator, and operating the first fractionator to fractionate the second input stream, the first reflux stream, and the second reflux stream, thereby forming a liquid product stream and a first vapor effluent stream in the first fractionator. The process includes directing the first vapor effluent stream into a second fractionator, directing the second input stream into the second fractionator at a location that is above the location where the first vapor effluent stream is input into the second fractionator, and operating the second fractionator to fractionate the second input stream and the first vapor effluent stream, thereby forming a second liquid product stream and a second vapor effluent stream in the second fractionator. The process includes directing the second liquid product stream into the first fractionator and fractionating the second liquid product stream.

Some embodiments of the present disclosure include a process for separating a natural gas stream. The process includes receiving an input stream of natural gas, separating the input stream into a first input stream and a second input stream, and cooling and separating the first input stream into a first reflux stream and a second reflux stream. The process includes directing the first reflux stream into a first fractionator, and directing the second reflux stream into the first fractionator at a location that is below a location where the first reflux stream is input into the first fractionator. The process includes directing the second input stream into a reflux drum and operating the reflux drum to form a bottom liquid stream and a top vapor stream. The process includes directing the bottom liquid stream and the top vapor stream into the first fractionator, and operating the first fractionator to fractionate the bottom liquid stream, the top vapor stream, the first reflux stream, and the second reflux stream, thereby forming a liquid product stream and a first vapor effluent stream in the first fractionator.

Some embodiments of the present disclosure include a process for separating a natural gas stream. The process includes receiving an input stream of natural gas, separating the input stream into a first input stream and a second input stream, and cooling and separating the first input stream into a first reflux stream and a second reflux stream. The process includes directing the first reflux stream into a first fractionator, and directing the second reflux stream into the first fractionator at a location that is below a location where the first reflux stream is input into the first fractionator. The process includes directing the second input stream into a reflux drum and operating the reflux drum to form a bottom liquid stream and a top vapor stream. The process includes directing the bottom liquid stream into the first fractionator, and operating the first fractionator to fractionate the bottom liquid stream, the first reflux stream, and the second reflux stream, thereby forming a liquid product stream and a first vapor effluent stream in the first fractionator. The process includes directing the top vapor stream and the first vapor effluent stream into a second fractionator, and operating the second fractionator to fractionate the top vapor stream and the first vapor effluent stream, thereby forming a second liquid product stream and a second vapor effluent stream in the second fractionator. The process includes directing the second liquid product stream into the first fractionator and fractionating the second liquid product stream.

Some embodiments of the present disclosure include a system for separating a natural gas stream. The system includes a natural gas inlet and a first fractionator. The first fractionator includes one or more inlets, a first vapor effluent outlet, and a first liquid product outlet. The system includes a chiller and a separator configured to cool and separate natural gas. The chiller and the separator are in fluid communication between the natural gas inlet and the first fractionator. A first fluid path is defined between the first fractionator and the natural gas inlet that passes through the chiller and the separator. A second fluid path is defined between the first fractionator and the natural gas inlet that bypasses the chiller and the separator.

Some embodiments of the present disclosure include a method of retrofitting a natural gas separation plant that includes a natural gas inlet, a first fractionator, a chiller, and a separator. The natural gas inlet is in fluid communication with the first fractionator along a first fluid path that passes through the chiller and the separator. The method includes fluidly coupling the natural gas inlet with the first fractionator along a second fluid path that bypasses the chiller and the separator.

### BRIEF DESCRIPTION OF THE DRAWINGS

So that the manner in which the features and advantages of the systems, configurations, constructions, apparatus, process, techniques, and methods of the present disclosure may be understood in more detail, a more particular description may be had by reference to specific implementations that are illustrated in the appended drawings. It is noted, however, that the drawings are simplified schematics and flow diagrams showing specific implementations for illustration and are, therefore, not to be considered limiting of the disclosed concepts, which include other effective applications as well. It is noted, for example, that certain applications may employ less than all of the different aspects described below.

FIG. 1A is a schematic and flow diagram illustrating a prior art system for natural gas processing, including the recovery of propane and heavier hydrocarbons from a natural gas stream.

FIG. 1B is a schematic and flow diagram illustrating another prior art system for natural gas processing including a recycle split vapor process.

FIG. 2 is a schematic and flow diagram illustrating a system for natural gas processing with a portion of an inlet fluid stream provided as an additional reflux stream to a fractionator in accordance with embodiments of the present disclosure.

FIG. 3 is a schematic and flow diagram illustrating a system for natural gas processing with a portion of an inlet fluid stream combined with a portion of a residue gas stream and provided as an additional reflux stream at a point in a fractionator above another reflux stream in accordance with embodiments of the present disclosure.

FIG. 4 is a schematic and flow diagram illustrating a system for natural gas processing with a portion of an inlet fluid stream combined with a portion of a residue gas stream and provided as an additional reflux stream at a point in a fractionator below another reflux stream in accordance with embodiments of the present disclosure.

FIG. 5 is a schematic and flow diagram illustrating a system for natural gas processing with a portion of an inlet fluid stream combined with a portion of a residue gas stream and with a reflux stream that is fed into a fractionator in accordance with embodiments of the present disclosure.

FIG. 6 is a schematic and flow diagram illustrating a system for natural gas processing that is retrofitted with additional components and piping, including a second fractionator, to provide a portion of an inlet fluid stream combined with a portion of a residue gas stream as an additional reflux stream in a fractionator in accordance with embodiments of the present disclosure.

FIG. 7 is a schematic and flow diagram illustrating another system for natural gas processing that is retrofitted with additional components and piping, including a reflux drum, to provide a portion of an inlet fluid stream combined with a portion of a residue gas stream as an additional reflux stream in a fractionator in accordance with embodiments of the present disclosure.

FIG. 8 is a schematic and flow diagram illustrating another system for natural gas processing that is retrofitted with additional components and piping, including a second fractionator and reflux drum, to provide a portion of an inlet fluid stream combined with a portion of a residue gas stream as an additional reflux stream in a fractionator in accordance with embodiments of the present disclosure.

FIG. 9 is a graph of recoveries in natural gas processing plants.

FIG. 10 is a schematic and flow diagram illustrating a system for natural gas processing, including the recovery of propane and heavier hydrocarbons from a natural gas stream, in accordance with embodiments of the present disclosure.

FIG. 11 is a schematic and flow diagram illustrating an alternative system for natural gas processing, including the recovery of propane and heavier hydrocarbons from a natural gas stream, in accordance with embodiments of the present disclosure.

FIG. 12 is a schematic and flow diagram illustrating an alternative system for natural gas processing, including the recovery of propane and heavier hydrocarbons from a natural gas stream, in accordance with embodiments of the present disclosure.

FIG. 13 is a schematic and flow diagram illustrating an alternative system for natural gas processing, including the recovery of propane and heavier hydrocarbons from a natural gas stream, in accordance with embodiments of the present disclosure.

FIG. 14 is a schematic and flow diagram illustrating an alternative system for natural gas processing, including the recovery of propane and heavier hydrocarbons from a natural gas stream, in accordance with embodiments of the present disclosure.

FIG. 15 is a graph propane recovery (y-axis) and ethane recovery (x-axis) for different processes; and

FIG. 16 is a graph of HP/MMscfd (y-axis) and % ethane recovery (x-axis) for different processes.

### DETAILED DESCRIPTION

The present disclosure includes systems and methods for the separation of fluid streams containing hydrocarbons, including the separation of ethane, propane, butane and/or heavier hydrocarbons from NGL and LPG streams. Natural gas liquids (NGLs) contain propane, butane, and other hydrocarbons. NGLs can have a higher value as one or more separate products and are, thus, often separated from natural gas streams. Moreover, reducing the concentration of higher hydrocarbons and water in the stream reduces or prevents the formation of hydrocarbon liquids and hydrates in pipelines carrying natural gas.

Once NGL is removed from a natural gas stream, the NGL can be fractionated into various constituents, such as ethane, propane, butane, and other hydrocarbons, which can, optionally, be sold as relatively high-purity products. Fractionation of NGL can be performed at a centralized processing plant where the NGL is removed from the natural gas stream, or can be performed downstream, such as in a regional NGL fractionation center. Fractionation includes heating a mixed NGL stream and processing this NGL stream through a series of distillation towers. The fractionation process may require passing the NGL stream through a series of distillation columns (or towers), and relying on the differences in the boiling points of the constituents to separate out various components of the NGL stream into discrete streams (product streams). For example, an initial NGL stream may be directed into a first distillation column of a first series of distillation columns, where the initial NGL stream is heated such that the lightest (lowest boiling point) component(s) of the NGL stream boil within the first distillation column(s) and exit the first distillation column(s) as a first overhead vapor. Thus, the lightest, lowest boiling point components of the NGL stream are separated from the remainder of the NGL stream in the first distillation column(s). The first overhead vapor is then condensed to form a first product stream containing the lightest, lowest boiling point components of the NGL stream, which are stored in a first product storage (e.g., a tank). A portion of the condensed, first overhead vapor can be used as a reflux in the first distillation column(s), and a remaining portion of the condensed, first overhead vapor is stored as a first product stream. By passing the NGL stream through the first distillation column(s), the weight percent of some components within the NGL stream is reduced. For example, if it is desired to target removal of a first component (e.g., methane), the NGL stream exiting the first distillation column(s) will contain more, or a higher concentration, of the first component than the initial NGL stream (fed into the first distillation column(s)). The portion or remainder of the NGL stream that does not boil in this first distillation column(s) will contain more of the relatively heavier components than the initial NGL stream, and is then passed from the bottom of the first distillation column(s) to a second distillation column(s), where the process is repeated to form a second product stream. The produced second product stream contains a higher concentration of the relatively heavier NGL components than the previous or first product stream. The process of passing the NGL stream through distillation columns can be repeated a desired number times until the desired product streams are extracted from the NGL stream. Each successive distillation column can extract a product stream that contains higher concentrations of relatively heavier components, than the products streams from the prior or upstream distillation column. Notably, the product streams from a distillation column may be passed through an additional distillation column or the same distillation column for additional refinement of the products, as desired.

### Natural Gas Liquids Processing

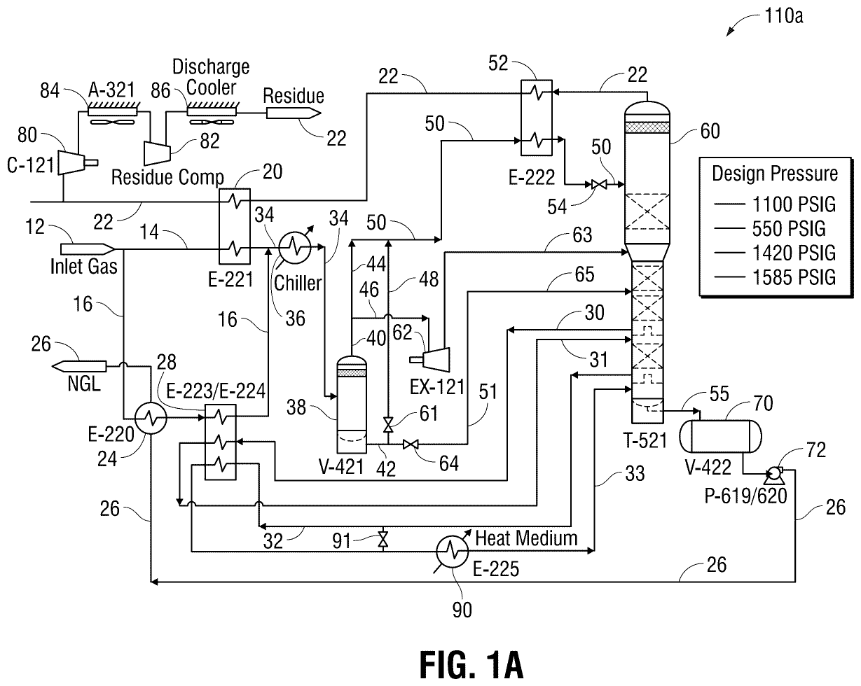

FIGS. 1A and 1B depict natural gas processing and recovery systems in accordance with the state of the art, to which the present disclosure relates. To illustrate and highlight certain concepts and structures introduced herein, various systems, subsystems, methods, processes, subprocesses, techniques, configurations, and constructions are introduced in the context of, or as modifications or improvements to, the systems and processes of FIGS. 1A and 1B. The present disclosure relates to and, in some respects, builds upon the disclosures of U.S. Provisional Patent Application No. 63/166,179 ('179 Application); United States Patent Publication No. 2022/0306553 ('553 Publication); and International Publication No. WO/2022/204563 ('563 Publication); each of which provides background relevant to aspects of the present disclosure. The entireties of the '179 Application, the '553 Publication, and the '563 Publication are incorporated herein by reference, for all purposes, and made a part of the present disclosure.

With reference to FIG. 1A (reproduced from FIG. 1 of the '553 Publication, with modifications), expander-based NGL recovery plant 110a is configured to separate ethane and propane from a gas stream using a Gas Subcooled Process (GSP). Plant 110a receives inlet fluid stream 12, sometimes referred to as a feed stream. Inlet fluid stream 12 is a natural gas fluid stream that contains a mixture of compounds including methane, ethane, propane, butane, and other hydrocarbons. Inlet fluid stream 12 contains compounds that are desirable in a product stream 26 of the plant 110a, such as ethane and/or propane, and may also contain compounds that are undesirable in the product stream 26 of the plant 110a, such as hydrocarbons other than ethane and propane. For example, the presence of some compounds in the product stream 26, at least above a certain concentration level within the product stream 26, may cause the product stream 26 to not meet certain specifications. In some embodiments, the product stream 26 is an NGL stream. The plant 110a is configured to separate out compounds from the inlet fluid stream 12 to produce the product stream 26, such that the product stream 26 does not contain the undesirable compounds, or at least contains a concentration of the undesirable compounds that is below a certain threshold concentration. Additionally, the plant 110a can be configured to dehydrate compounds in the inlet fluid stream 12 to prevent hydrate (ice) formation under cryogenic conditions. In some embodiments, inlet fluid stream 12 is entirely in the gas phase, or at least substantially in the gas phase.

The inlet fluid stream 12 is split into two streams, including a first fluid stream 14 and a second fluid stream 16. First fluid stream 14 is directed through a first heat exchanger 20. Within the first heat exchanger 20, the first fluid stream 14 is passed in thermal communication with a residue gas stream 22, such that heat transfer occurs between the first fluid stream 14 and the residue gas stream 22. Passing the first fluid stream 14 through the first heat exchanger 20 cools the first fluid stream 14, such that the first fluid stream 14 exits the first heat exchanger 20 at a lower temperature than a temperature of the first fluid stream 14 at entry into the first heat exchanger 20. Residue gas stream 22 is heated within first heat exchanger 20.

The second fluid stream 16 is directed through a second heat exchanger 24, where it is passed in thermal communication with the product stream 26. Within the second heat exchanger 24, heat transfer occurs between the second fluid stream 16 and the product stream 26, such that the second fluid stream 16 cools and exits the second heat exchanger 24 at a lower temperature than at entry into the second heat exchanger 24. Product stream 26 is heated within second heat exchanger 24.

The second fluid stream 16 exiting the second heat exchanger 24 then flows through a third heat exchanger 28. Within the third heat exchanger 28, the second fluid stream 16 is in thermal communication with a first reboiler stream 30 and a second reboiler stream 32 from a fractionator 60, such that heat transfer occurs amongst the second fluid stream 16, the reboiler stream 30, and the reboiler stream 32. Passing the second fluid stream 16 through the third heat exchanger 28 cools the second fluid stream 16, such that upon exiting the third heat exchanger 28 the second fluid stream 16 is at a lower temperature than upon entering the third heat exchanger 28. Streams 30 and 32 are both heated within third heat exchanger 28. While heat exchange amongst the second fluid stream 16, reboiler stream 30, and reboiler stream 32 is depicted as occurring in a single heat exchanger, the heat exchange may be achieved through separate heat exchangers (e.g., in series or parallel). The contents of the reboiler streams 30 and 32 can vary depending on the amount of ethane that is being rejected. Stripping vapors 31 and 33 are formed by passing the reboiler streams 30 and 32 through the second heat exchanger 28 and heating the reboiler streams 30 and 31 therein. The stripping vapors 31 and 33 strip methane and ethane (in rejection mode) within the fractionator 60. The stripping vapor 31 can contain, for example, C₁ (e.g., 44%), C₂ (e.g., 23%), C₃ (e.g., 18%), C₄ (e.g., 9%), <2% inerts, and a remainder of C₅ or heavier (all mole %). The stripping vapor 33 can contain, for example, C₁ (e.g., 0.7 to 4%), C₂ (e.g., 45 to 55%), C₃ (e.g., 35 to 43%), C₄ (e.g., 6 to 7%), <0.7% inerts, and a remainder C₅ or heavier components (all mole %). In some embodiments, the stripping vapors 31 and 33 are fully vaporized. In other embodiments, the stripping vapors 31 and 33 are not be fully vaporized.

First and second fluid streams 14 and 16 are then recombined to form combined stream 34. While the first and second fluid streams 14 and 16 are shown as being separated, separately cooled, and then recombined, the inlet fluid stream 12 can be cooled without being separated.

The combined stream 34 passes through and is cooled by chiller 36. The cooled combined stream 34 then passes into separator 38 (e.g., a cold separator), where the combined stream 34 is separated into a vapor stream 40 and a condensed liquid stream 42. The contents of the vapor stream 40 and the condensed liquid stream 42 are, at least partially, dependent on the composition of the inlet fluid stream 12 and the temperature and pressure of the separator 38. In one example, the vapor stream 40 contains from 80 to 85% by mole methane and inert components, from 9 to 12% by mole ethane, and about 3.5% by mole propane and a minor amount of heavies. In one example, the condensed liquid stream 42 contains from 40 to 45% by mole methane, from 1 to 2% by mole inert components, from 20 to 25% by mole ethane, from 15 to 20% by mole propane, from 5 to 10% by mole butane, and about 4% by mole heavies.

The vapor stream 40 is discharged from the separator 38 and is divided into a first vapor stream 44 and a second vapor stream 46. The condensed liquid stream 42 is discharged from the separator 38 and is divided into a first liquid stream 48 and a second liquid stream (third reflux stream 51). The first liquid stream 48 passes through valve 61 and is then combined with the first vapor stream 44, forming a first reflux stream 50. First reflux stream 50 is passed through a fourth heat exchanger 52. Within the fourth heat exchanger 52, the first reflux stream 50 is in thermal communication with the residue gas stream 22, such that heat transfer occurs between the first reflux stream 50 and the residue gas stream 22. Passing the first reflux stream 50 through the fourth heat exchanger 52 cools the first reflux stream 50, such that the first reflux stream 50 exits the fourth heat exchanger 52 at a lower temperature than a temperature of the first reflux stream 50 at entry of the fourth heat exchanger 52. Residue gas stream 22 is heated in fourth heat exchanger 52. Additionally, within the fourth heat exchanger 52, at least a portion of the first reflux stream 50 is condensed. The condensed first reflux stream 50 then passes through expansion valve 54. Within expansion valve 54, the condensed first reflux stream 50 is flash expanded to a pressure above an operating pressure of the fractionator 60. After exiting the expansion valve 54, the first reflux stream 50 is supplied to the fractionator 60.

The second vapor stream 46 is passed to an expander-booster compressor combination, or turboexpander 62. Within the turboexpander 62, mechanical energy is extracted from the relatively high-pressure feed of the second vapor stream 46. The turboexpander 62 expands the second vapor stream 46 such that the second vapor stream 46 is brought to a pressure that is within the range of the operating pressure(s) of the fractionator 60. Within turboexpander 62, the second vapor stream 46 is also cooled, reducing a temperature of the second vapor stream 46 and forming a second reflux stream 63. The second reflux stream 63 is supplied to the fractionator 60 at a position that is below the position where the first reflux stream 50 is supplied to the fractionator 60.

A portion of stream 42 forms the third reflux stream 51, which is directed through an expansion valve 64 such that the pressure of the third reflux stream 51 is lowered to a pressure that is within the range of the operating pressure(s) of the fractionator 60, while also cooling the third reflux stream 51. The third reflux stream 51 is supplied to the fractionator 60 at a position that is below the position where the second reflux stream 63 is supplied to the fractionator 60.

The fractionator 60 can operate as a demethanizer tower, and can be or include a conventional distillation column containing multiple, vertically spaced trays, one or more packed beds, or combinations thereof. Within the fractionator 60, components in the vapor phase rise upward and relatively colder components in the liquid phase fall downward. The trays and/or packing in the fractionator 60 provide for contact between vapor phase compounds (e.g., vapor phase within the second reflux stream 63) within the fractionator 60 rising upward and liquid phase compounds within the fractionator 60 falling downward, such that ethane, propane, butane, and heavier components condense and are absorb into the liquid phase within the fractionator 60. While the fractionator 60 is described as having a top, bottom, middle, lower, higher and other vertical, positional sections, one skilled in the art would understand that these designations and conventions have functional and processing relevance and do not limit the precise arrangement of the fractionators.

First and second reboiler streams 30 and 32 of liquids are drawn from fractionator 60 and directed to the heat exchanger 28. The heat exchanger 28 heats and vaporizes the first and second reboiler streams 30 and 32 of liquids from fractionator 60, forming the stripping vapors 31 and 33, respectively, which are directed back into the fractionator 60. After passing through the heat exchanger 28, the stripping vapor 33 flows through a reboiler 90 to provide additional heat to the stripping vapor 33. The stripping vapors 31 and 33 flow upwards within a column of the fractionator 60 and strip liquid that is flowing downward in the fractionator 60 such that the stripping vapors 31 and 33 remove methane and lighter components from the liquid. Plant 110a includes a valve 91 connecting the second reboiler stream 32 with the stripping vapor 33 stream such that at least a portion of the reboiler stream 32 can bypass the heat exchanger 28 and/or such that at least a portion of the stripping vapor 33 can be recycled back through the heat exchanger 28.

Fractionation of the inputs into the fractionator 60 form a liquid product 26 and a vapor product, residue gas stream 22. The liquid product 26 is collected at a bottom of the fractionator 60 and discharged to a natural gas surge tank 70. The product stream 26 is discharged (e.g., pumped) from the natural gas surge tank 70, such as via use of a booster pump 72. For example, the product stream 26 can be pumped to storage, transport, or another location. Prior to exiting the plant 110a, the product stream 26 flows through the second heat exchanger 24 to cool the second fluid stream 16.

The residue gas stream 22 exists a top of the fractionator 60 as a vapor phase. The residue gas stream 22 passes through the fourth heat exchanger 52 to exchange heat with the first reflux stream 50 and, downstream therefrom, passes through the first heat exchanger 20 to exchange heat with the first fluid stream 14. The residue gas stream 22 is then re-compressed in two stages via compressors 80 and 82. The residue gas stream 22 passes through compressor 80, through cooler 84, through compressor 82, and then through cooler 86. After being compressed and cooled, the residue gas stream 22 is discharged from the plant 110a.

### Recycle Split Vapor Process

The system of FIG. 1B, plant 110b is substantially similar to plant 110a of FIG. 1A. However, in plant 110b the residue gas stream 22 is split in a recycle split vapor (RSV) process into a first residue gas stream 22a and a second residue gas stream 22b after passing through the cooler 86. The first residue gas stream 22a is discharged from the plant 110b, as is done with residue gas stream 22 in the process of FIG. 1A. The second residue gas stream 22b is directed through the first heat exchanger 20 to exchange heat with the residue gas stream 22 existing the fractionator 60 and with the first fluid stream 14. The second residue gas stream 22b is directed from the first heat exchange 20 to the fourth heat exchanger 52 to exchange heat with the residue gas stream 22 exiting the fractionator 60 and with the first reflux stream 50. The second residue gas stream 22b is directed from the fourth heat exchanger 52, through valve 25 (e.g., expansion valve) and into the fractionator 60. As shown in FIG. 1B, the fractionator 60 includes multiple beds, including first bed 69a, second bed 69b, and beds 67a-67c.

### Natural Gas Liquids Recovery System—Ethane Recovery

For ethane recovery, the inlet fluid stream 12 is separated into ethane and heavier liquids, referred to as NGL (i.e., product stream 26), and methane, referred to as residue or sales gas (i.e., residue gas stream 22). The process can begin with a filtered dry inlet gas from a molecular sieves dehydration system as the inlet fluid stream 12. The inlet fluid stream 12 is split into two streams, fluid streams 14 and 16. The fluid stream 16 is temperature-controlled, through a series of cross-exchangers (i.e., exchangers 26 and 28) with other cold process streams, such as produced NGL (i.e., product stream 26) and tower liquids (i.e., reboiler streams 30 and 32), providing for tower re-boiling. The fluid stream 16 can, optionally, be passed through a mechanical refrigeration package chiller (e.g., chiller 36). The fluid stream 16 is then directed into cold separator 38. The fluid stream 14 flows through a gas/gas exchanger (i.e., exchanger 20) that cools the fluid stream 14 prior to the fluid stream 14 being recombined with the fluid stream 16 in either the mechanical refrigeration package chiller 36 and/or the cold separator 38 in which the gas and liquid phases of the streams 14 and 16 are separated.

The cold separator 38 scrubs and separates the fluid streams 14 and 16, which can prevent potentially damaging liquids from entering an inlet of the expander side of the turboexpander 62. From the cold separator 38, a portion of the vapors (i.e., stream 44) and liquids (i.e., stream 48) are combined into the first reflux stream 50 and then cooled through a reflux condenser as a “pseudo-reflux” stream by flashing stream 50 (GSP reflux stream) into a secondary rectification (packed bed) section of the demethanizer tower (i.e., fractionator 60). The flashed stream 50 provides cooling that will tend to hold down ethane in the tower of the fractionator 60 and enhance the recovery potential of the cryogenic system. Vapors from the cold separator 38 are routed through a Joule-Thompson valve and/or an expander side of the turboexpander 62 prior to entering the side of demethanizer tower below a second packed bed (e.g., as second reflux stream 63), providing additional cooling by expanding two-phase gas. Pressure in the demethanizer tower is, generally, driven down to provide higher efficiency in the separation of methane, increasing the overall recovery of ethane. The remaining liquids from the cold separator 38 flow to a middle of the demethanizer tower (e.g., as third reflux stream 51).

The fractionator 60, functioning as a demethanizer tower, has two thermosiphons while operating in ethane recovery mode, including: (1) the demethanizer bottom reboiler (stream 32); and (2) the demethanizer side reboiler (stream 30). The demethanizer bottom reboiler stream 32 and demethanizer side reboiler stream 30 provide heat by cross-exchanging the inlet fluid stream 12 with a demethanizer bottom liquid draw and a demethanizer side liquid draw from the fractionator 60. The heating of the inlet fluid stream 12 reduces or avoids excessive methane accumulation in the product stream 26.

The fractionator 60 forms two product streams, including: (1) the residue sales gas stream 22 discharged from the top of the fractionator 60; and (2) the NGL product stream 26 discharged from the bottom of the fractionator 60. The residue gas stream 22 is the coldest prior to cross-exchange in the reflux condenser (exchanger 52) and the gas/gas exchanger (exchanger 20), both of which decrease the temperature of the residue gas stream 22. The residue gas stream 22 can have a tap for fuel gas, and can be directed through compressors 80 and 82 and coolers 84 and 86, where work is done compressing the residue gas stream 22 by increasing the pressure and temperature of the stream. The first residue gas stream 22a can be directed to custody transfer meters and pipeline pig launchers. A slip stream of the compressed residue gas stream, i.e., the second residue gas stream 22b, can be recycled, filtered to remove lube oil, and cross-exchanged with the tower overheads in the reflux condenser and the gas/gas exchanger, and then expansion cooled through a Joule-Thompson valve to provide a top lean reflux stream 25 (RSV reflux) to the demethanizer tower. The reflux stream 25 limits the loss of ethane as residue gas. The recovered NGL liquids, product stream 26, are collected in the demethanizer surge tank 70, which is equalized with the demethanizer tower. The product stream 26 is then boosted in pressure by the pipeline booster pump(s) 72 prior to being sent to a facilities product pipeline pumps, custody transfer meters, and pipeline pig launchers.

In some embodiments, in ethane recovery mode, from 90 to 99% ethane of inlet stream 12 is recovered in the liquid product stream 26.

### Natural Gas Liquids Recovery System—Ethane Rejection

For ethane rejection, the inlet fluid stream 12 is separated into propane and heavier liquids, referred to as NGLs, and a combination of methane and ethane, referred to as residue or sales gas. Ethane rejection follows, generally, the same flow scheme as described above with respect to ethane recovery, but with some differences, as noted below. For ethane rejection, the majority of ethane is driven out the top of the demethanizer tower in residue gas stream 22, where propane and heavier hydrocarbons are liquefied and recovered in product stream 26. The driving force for the separation between ethane and propane is added heat and higher tower pressures in the tower of the fractionator 60. The heat provided by the demethanizer bottom reboiler, stream 32, in the ethane recovery is replaced by a trim reboiler in the ethane rejection mode. Heat provided by a demethanizer side reboiler is used in the ethane rejection mode; however, the source of the stream is replaced by liquids from the cold separator 38 in lieu of the latent heat transfer siphoned from the fractionator 60. With the added heat to the fractionator 60, a deethanized product cooler is added to the booster pump discharge 72 prior to the product stream 26 being sent to the facilities product pipeline pumps, custody transfer meters, and pipeline pig launchers.

In some embodiments, in ethane rejection mode, from 0 to 20% ethane from inlet stream 12 is recovered in the liquid product stream 26, with the remainder exiting the fractionator 60 in the residue gas stream 22.

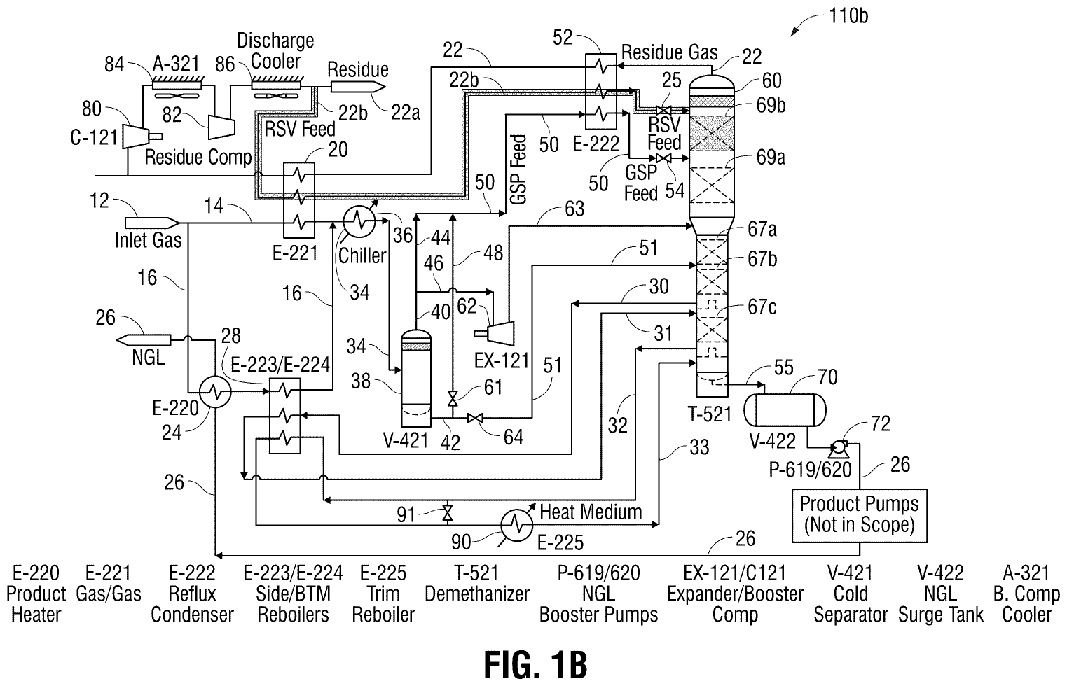

The above descriptions of FIGS. 1A and 1B describe prior art systems and processes for NGL processing and recovery, which are modified and/or improved upon as provided in the present disclosure and illustrated through the exemplary systems and methods of FIGS. 2-8 and the accompanying descriptions.

### Inlet Fluid Reflux Stream

Some embodiments include providing a portion of the inlet fluid stream as an additional reflux stream into the fractionator. With reference to FIG. 2, plant 210 is similar to plant 110a, with like reference numerals designating like parts.

As with FIGS. 1A and 1B, in plant 210 the inlet fluid stream 12 is a fluid stream of natural gas containing a mixture of compounds including methane, ethane, propane, butane, and other hydrocarbons. However, at the inlet of the plant 210, the inlet fluid stream 12 is separated into three separate fluid streams rather than two fluid streams. The inlet fluid stream 12 is separated into a first fluid stream 14, a second fluid stream 16, and a third fluid stream 18. The contents of each of the fluid streams 14, 16, and 18 are identical. The amount of inlet fluid stream 12 that is diverted as third fluid stream 18 depends, at least partially, on the particular design of the plant 210. In one example, the amount of third fluid stream 18 fed into plant 210 is from greater than 0 to 40 million standard cubic feet per day (mmscfd), from greater than 0 to 20 mmscfd, from 20 to 40 mmscfd, less than 40 mmscfd, less than 20 mmscfd, or any range or discrete value therebetween. In some embodiments, from 10 to 15 vol. % of the inlet fluid stream 12 is diverted as the third fluid stream 18.

The first and second fluid streams 14 and 16 pass through and are processed within plant 210 in the same or substantially the same manner as described in reference to FIG. 1A. The third fluid stream 18 is directed to the first heat exchanger 20 to exchange heat with the residue gas stream 22 and the first fluid stream 14. Within the third heat exchanger 20, the third fluid stream 18 is cooled. After passing through the first heat exchanger 20, the third fluid stream 18 is directed to the fourth heat exchanger 52 to exchange heat with the residue gas stream 22 and the first reflux stream 50. Within the fourth heat exchanger 52, the third fluid stream 18 is cooled. After passing through the fourth heat exchanger 52, the third fluid stream 18 passes through expansion vale 25, which lowers the pressure of the third fluid stream 18. In some embodiments, the expansion valve 25 lowers the pressure of the third fluid stream 18 to a pressure that is equal to or substantially equal to a pressure of the fractionator 60.

The third fluid stream 18 is directed from the expansion valve 25 into the fractionator 60 as fourth reflux stream of the fractionator 60. The fourth reflux stream, third fluid stream 18, enters the fractionator 60 at a location that is above a location where the first reflux stream 50 enters the fractionator 60. The third fluid stream 18 enters the fractionator 60 at second bed 69b, and the first reflux stream 50 is enters the fractionator 60 at first bed 69a. Thus, the system and process of FIG. 2 includes an additional thermal exchange between a portion of the inlet fluid stream 12 and the affluent residue gas stream 22 from the fractionator 60, relative to FIG. 1A, which is then provided to the fractionator 60 as an additional reflux stream. In some embodiment, the performance of the system and process of FIG. 2 is similar to that of a GSP plant.

By diverting a portion of the inlet fluid stream 12, as third fluid stream 18, and directing the third fluid stream 18 into the fractionator 60, the plant 210 is provided with an increased natural gas processing capacity in comparison to being limited to processing only natural gas that passes through and is processed along the flow paths of the first and second fluid streams 14 and 16. That is, rather than constraining the flow paths of the first and second fluid streams 14 and 16 in the plant 210 with additional flow, additional flow in the form of the third fluid stream 18 is provided along a new flow path.

In some embodiments, the flow path of the additional third fluid stream 18 includes no processing upstream of the fractionator 60, or less processing than the processing of streams 14 and 16. That is, unlike the first and second fluid streams 14 and 16, the third fluid stream 18 bypasses any separation processing prior to being input into the fractionator 60. For example, the first and second fluid streams 14 and 16 pass through separator 38, whereas, the third fluid stream 18 is not passed through the separator 38 and is not otherwise processed to separate a liquid phase from a vapor phase upstream of the fractionator 60. In some embodiments, the only processing of the third fluid stream 18 upstream of the fractionator 60 is cooling the third fluid stream 18. The additional input capacity provided to the plant 210 by the third fluid stream 18 provides for an increase of the rate of volume (e.g., volume/time) of natural gas that the plant 210 can process. The additional increase in input capacity by using third fluid stream 18 can be attained without requiring a larger plant.

In some embodiments, the third fluid stream 18 is used in leu of the recycle residue gas stream 22b of FIG. 1B. However, in other embodiments, the third fluid stream 18 is used in combination with a recycle residue gas stream.

### Inlet Fluid and Residue Gas Reflux Stream

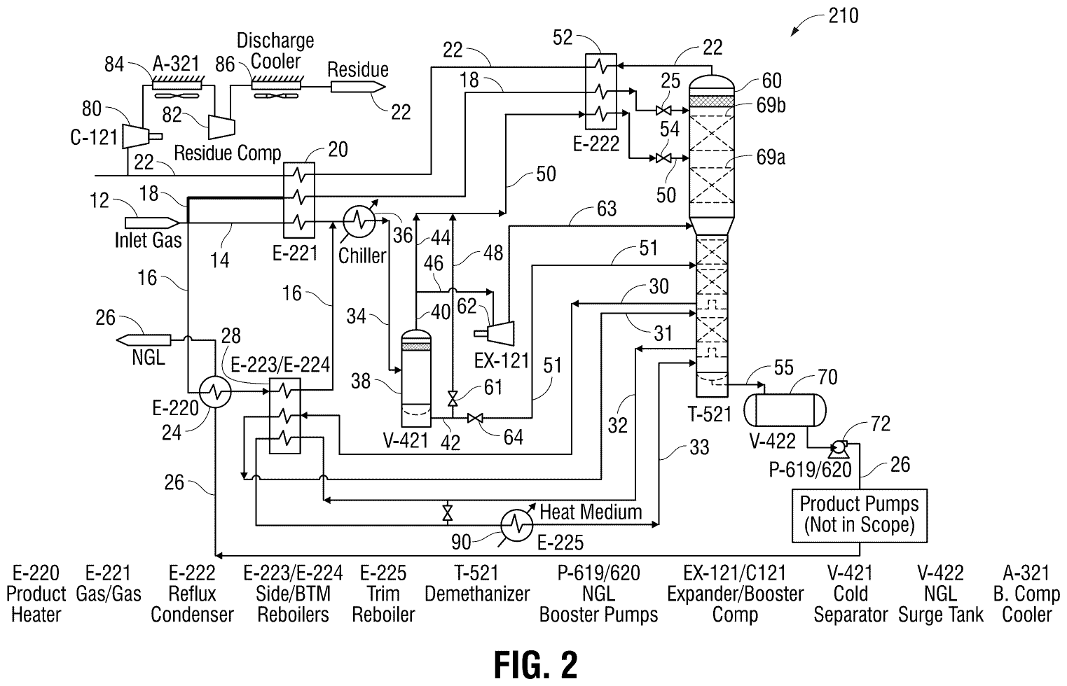

Some embodiments include combing a portion of the inlet fluid stream with a portion of the residue gas stream and providing this combined stream as a reflux stream into the fractionator. With reference to FIG. 3, plant 310 is substantially similar to plant 110b and plant 210, with like reference numerals designating like parts. In some respects, plant 310 is a combination of the concepts of plant 110b and the plant 210 into a single plant.

In plant 310, the inlet fluid stream 12 is separated into the three separate fluid streams, including first fluid stream 14, second fluid stream 16, and third fluid stream 18. The first and second fluid streams 14 and 16 are processed in the same or substantially same manner as in FIG. 2. However, the third fluid stream 18 is combined with second residue gas stream 22b, forming fourth reflux stream 318. The fourth reflux stream 318 is directed to the first heat exchanger 20 to exchange heat with the residue gas stream 22 and the first fluid stream 14, cooling the fourth reflux stream 318. After passing through the first heat exchanger 20, the fourth reflux stream 318 is directed to the fourth heat exchanger 52 to exchange heat with the residue gas stream 22 and the first reflux stream 50, further cooling the fourth reflux stream 318. After passing through the fourth heat exchanger 52, the fourth reflux stream 318 passes through expansion vale 25 where the pressure of the fourth reflux stream 318 is lowered (e.g., to the pressure of the fractionator 60). The fourth reflux stream 318 is then directed from the expansion vale 25 into the fractionator 60. In the embodiment shown in FIG. 3, the fourth reflux stream 318 enters the fractionator 60 at a location that is above where the first reflux stream 50 enters the fractionator 60.

In some embodiments, a system and process in accordance with FIG. 1B can be modified to form a system and process in accordance with FIG. 3. For example, a three-pass exchanger (e.g., exchanger 20) for an RSV process can be modified by adding a tap set that allows for the pass to be filled with a portion of the inlet fluid stream 12 instead of the residue recycle gas (RSV, stream 22b).

A plant configured for an RSV process (e.g., as shown in FIG. 1B) has higher propane stability in comparison to a plant configured for a GSP process (e.g., as shown in FIG. 1A). Providing a portion of the inlet fluid stream 12 as a reflux allows for more nameplate, which may be advantageous when capacity is key. The use of a portion of the inlet fluid stream 12, third fluid stream 18, allows for more of the inlet fluid stream 12 to be input into the plant 310 without providing more of the inlet fluid stream 12 into the typical inlet side of the plant 310 (i.e., as is done with fluid streams 14 and 16). This added input capacity of the plant 310 for the inlet fluid stream 12 can be from 10% to 20% of nameplate, for example. This added capacity is advantageous to processors that have a fixed-fee contract or are paid based on the rate of inlet fluid stream 12 that is provided to plant.

The plant 310 is configured to allow for a shift from C₂ recovery of between 92% and 99%, depending on added capacity (92% performance) versus added recovery (99% performance). The C₃ recovery of the plant 310 is more stable in RSV mode, particularly in ethane rejection mode, which can be advantageous. The plant 310 provides the ability to tradeoff between recoveries and capacities of these processes. In some embodiments, the increased input capacity of the plant provided by using a portion of the inlet fluid stream 12 as a reflux stream can at least partially reduce recoveries of desired products. However, by mixing a portion of the inlet fluid stream 12 with a portion of the residue gas stream 22b (i.e., forming stream 318), the advantages of increased input capacity can be balanced with the advantages of superior recoveries.

In the embodiments of FIGS. 2 and 3, the reflux streams 18 and 318, each containing a portion of the inlet fluid stream 12, are directed into a top bed 69b of the fractionator 60, above the first reflux stream 50 (i.e., GSP feed), which is the bed that an RSV stream (stream 22b shown in FIG. 1B) would typically be fed. In the event of blending a portion of the inlet fluid stream 12 with a portion of the residue gas stream 22b (as shown in FIG. 3), feeding the reflux stream 318 at the top bed 69b provides additional recovery capabilities operating between GSP and RSV configurations.

While the fourth reflux stream 318 is shown in FIG. 3 as being input into the fractionator 60 at a point above the first reflux stream 50, the systems and processes disclosed herein are not limited to this point of input and may include inputting the additional reflux stream at the same point as the first reflux stream 50 or at a point that is below the first reflux stream 50.

### Combined Reflux Stream Input Variations

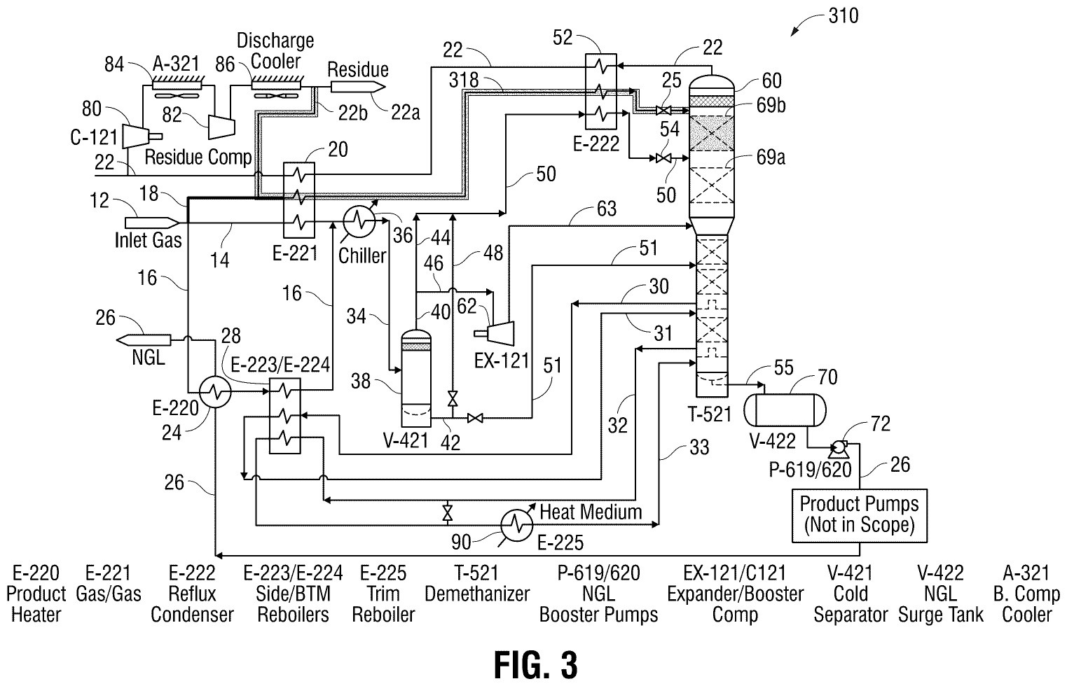

With reference to FIG. 4, plant 410 is substantially similar to plant 310, with like reference numerals designating like parts. However, in plant 410 the additional reflux stream, fourth reflux stream 418, enters the fractionator 60 at a point below the first reflux stream 50 rather than above (e.g., as with stream 318). Thus, in plant 410 rather than the fourth reflux stream 418 being fed into the top bed 69b of the fractionator 60, the fourth reflux stream 418 is fed into the first bed 69a of the fractionator 60, below the top bed 69b, and the first reflux stream 50 is fed into the top bed 69b of the fractionator 60 above the fourth reflux stream 418. The fourth reflux stream 418 contains a portion of the inlet fluid stream 12 without separation thereof, whereas, the first reflux stream 50 contains portions of the inlet fluid stream 12 that have been chilled and separated (e.g., in separator 38) such that it is a relatively leaner stream in comparison to the fourth reflux stream 418. Leaner reflux streams can enhance recoveries. As reflux stream 50 is a leaner stream than reflux stream 418 due to the removal of heavier hydrocarbons separated from the inlet via the separator 38, the reflux stream 50 facilitates the avoidance of desirable hydrocarbons being sent into the overhead (as stream 22). Switching the location of where the reflux streams are fed into the fractionator 60, such that the reflux stream 418 is input at a lower point in the fractionator 60 than the reflux stream 50 provides for a more stable processing that can achieve recoveries closer to those achieved by a GSP process

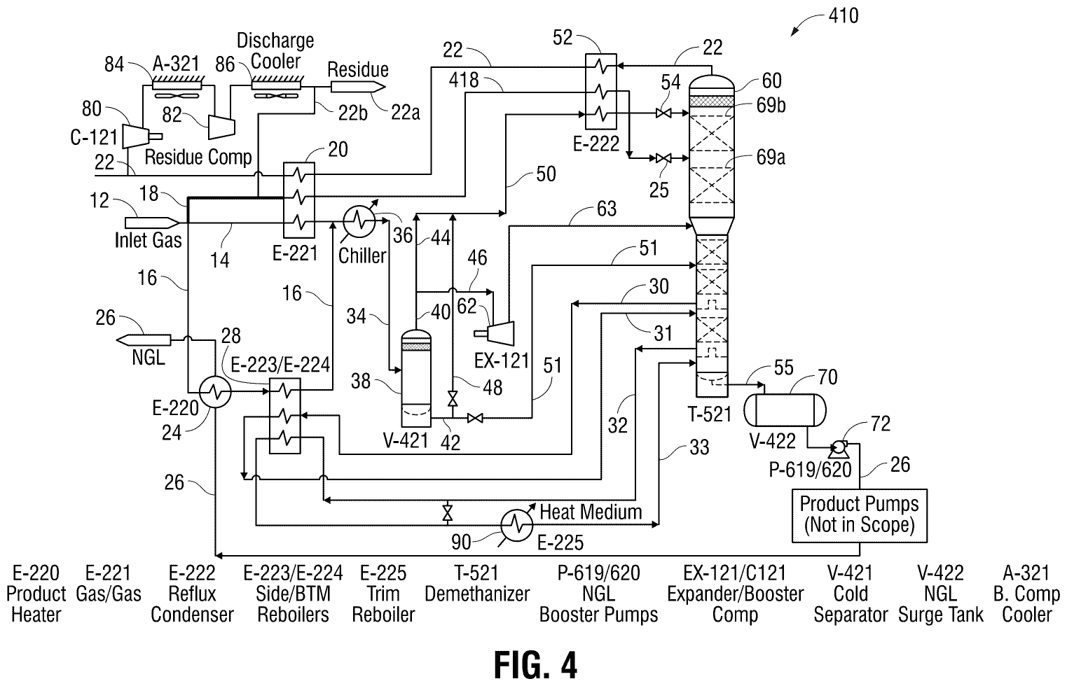

With reference to FIG. 5, plant 510 is substantially similar to plant 410, with like reference numerals designating like parts. However, in plant 510 the additional reflux stream, fourth reflux stream 518, is combined with the first reflux stream 50, forming combined reflux stream 519, prior to being input into the fractionator 60. Combined reflux stream 519 is then directed into the fractionator 60. Thus, in plant 510, the reflux streams 50 and 518 are blended after chilling and are then fed into the fractionator 60 at a common location. Combining the reflux streams 50 and 518 and feeding them into the fractionator 60 at a common location eliminates the need for an additional top bed (e.g., bed 69b) in the fractionator 60; thereby, simplifying the design of the fractionator 60 and providing a shorter fractionator 60 that is cheaper to construct.

In some embodiments, an existing GSP plant (e.g., as shown in FIGS. 1A and 1B) can be retrofitted with piping and a three-pass exchanger to form a system in accordance with embodiments of the present disclosure, provided that the residue gas line (stream 22) and turboexpander 62 of the existing system are capable handling the added flow to the plant with residue recompression.

Table 1, below, presents performance data for a 7.08 gas at 250 MMscfd inlet/nameplate.

TABLE 1

| | SMP | | | | | | | |
|---|---|---|---|---|---|---|---|---|
| | Recovered | Rejected | SMP-S | | SMP-E | | GSP | |
| | (Rec.) | (Rej.) | Rec. | Rej. | Rec. | Rej. | Rec. | Rej. |
| GPM | 7.08 | 7.08 | 7.08 | 7.08 | 7.08 | 7.08 | 7.08 | 7.08 |
| % C₂ | 91.92 | 10 | 92.06 | 10 | 90.32 | 10 | 92.1 | 10 |
| % C₃ | 99.38 | 90.74 | 99.49 | 91.53 | 99.37 | 90.54 | 99.54 | 91.4 |
| % iC₄ | 99.88 | 97.49 | 99.91 | 98.42 | 99.88 | 97.99 | 99.92 | 98.18 |
| Refrigeration | 7011 | 2892 | 7028 | 2911 | 6861 | 2892 | 6854 | 3454 |
| HP | | | | | | | | |
| Residue HP | 18446 | 19707 | 18438 | 19695 | 18510 | 19707 | 18891 | 20343 |

As shown in Table 1, in recovery mode the SMP-S configuration (i.e., the configuration of FIG. 4) provides the closest results to GSP recoveries, but with lower total HP. Also, in rejection mode the SMP-S configuration provides better recoveries than GPS recoveries, with lower total HP. The SMPE configuration (i.e., the configuration of FIG. 5) provides lower recovery than a GSP plant, but may still satisfy most performance contracts for GSP. The SMP configuration (i.e., the configuration of FIG. 2) provides performance slightly less than the SMPS configuration, but is an easier configuration to retrofit an existing RSV plant into. The concepts of FIGS. 4 and 5 of varying the location of input can be applied to the process scenarios of either FIG. 2 or 3.

### Retrofits

In some embodiments, an existing plant is retrofitted to perform one or more embodiments of the processes disclosed herein. For example, and existing plant can be retrofitted to include the components of the systems of any of FIGS. 2-5 and/or to perform the processes described in reference to any of FIGS. 2-5.

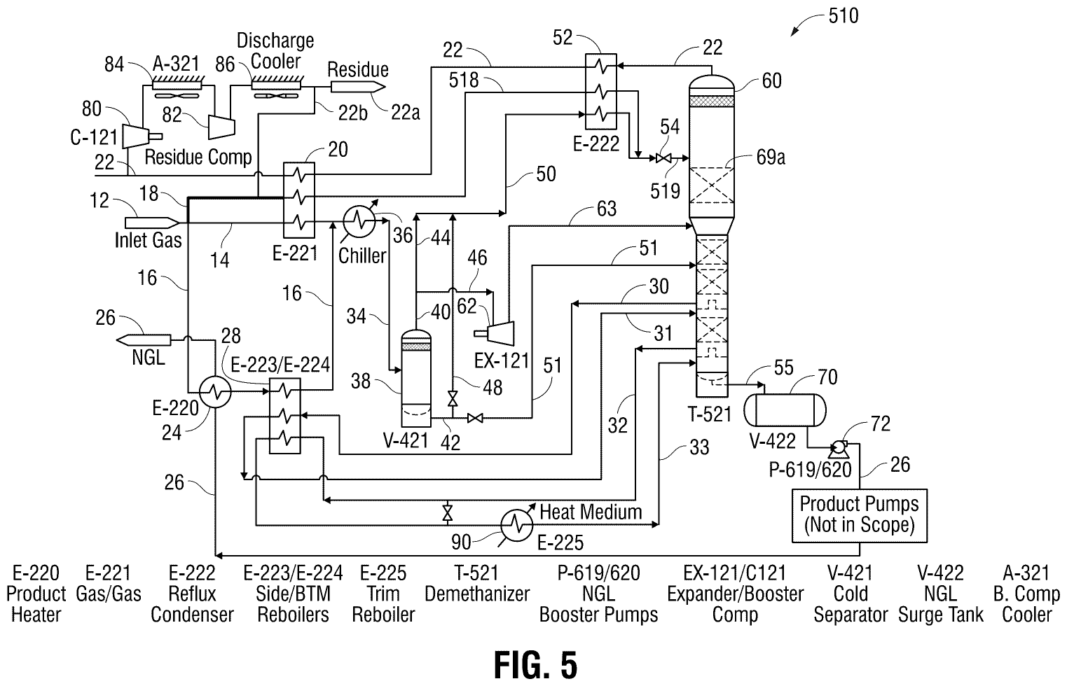

With reference to FIG. 6, plant 610 is similar to the plants shown and described in reference to FIGS. 2-5, with like reference numerals designating like parts. In plant 610, an existing GSP or industrial standard plant is modified to include a second fractionator 660, fifth heat exchanger 699, and pump 672. In addition to these components, the plant 610 is modified to include piping for the various additional flow lines required to provide streams to and from each of the second fractionator 660, fifth heat exchanger 699, and pump 672.

In plant 610, a portion of the inlet fluid stream 12, third fluid stream 18, is directed to be combined with a portion of the second residue gas stream 22b, forming combined reflux stream 618. Reflux stream 618 is then fed through fifth heat exchanger 699 to cool reflux stream 618, through expansion vale 697 to reduce a pressure of reflux stream 618, and into the second fractionator 660. Reside gas stream 22 from the fractionator 60 is also fed into the second fractionator 660 at a location below the feed point of the reflux stream 618. Within the second fractionator 660, a bottom liquid stream 655 is formed and is pumped, via pump 672 to be combined with stream 50, forming reflux stream 619 prior to passing through the expansion valve 54 and into the fractionator 60. The contents of bottom liquid stream 655 is affected by the contents of the inlet fluid stream 12, and can be leaned-out by capturing hydrocarbons in the second fractionator 660.

Within the second fractionator 660, a top vapor stream 622 is formed. Top vapor stream 622 is split into a first vapor stream 622a and a second vapor stream 622b. The first vapor stream 622a is directed through the fifth heat exchanger 699 to exchange heat with the reflux stream 618. The second vapor stream 622b is directed through the fourth heat exchanger 52 to exchange heat with stream 50 to cool stream 50. After the first and second vapor streams 622a and 622b pass through the fifth and fourth heat exchangers 699 and 52, respectively, the first and second vapor streams 622a and 622b are then recombined as the residue stream 622 and fed to the compressor 80, cooler 84, compressor 82, and cooler 86. In some embodiments, in ethane recovery mode, top vapor stream 622 contains methane and lighter hydrocarbons. In some embodiments, in rejection mode, top vapor stream 622 contains a blend of methane and ethane, with some ethane from the inlet gas stream 12 captured to avoid heating value issues in the residue gas. In one example, in ethane rejection mode, top vapor stream 622 is approximately 82% or more methane and 12% or less ethane.

The plant 610 can provide a similar performance in comparison to the SMP process of FIG. 2. The second fractionator 660 can operate at a lower pressure than the fractionator 60. If a pre-existing plant is an industrial standard plant without a GSP reflux, then the plant can be retrofitted to include both a GSP reflux stream and an SMP reflux stream in a single new fractionator tower. In the embodiment of FIG. 6, input capacity is increased and potential enhancements in recovery with the RSV residue blend in stream 619 are provided. The embodiment of FIG. 6 provides for a reduction in bottleneck strain on existing equipment and an increase in residue horse power (HP) and/or refrigeration HP.

The plant of FIG. 6, prior to the retrofitting to add the second fractionator 660, is designed and configured for GSP, such that the fractionator 60 does not have an RSV bed (i.e., does not have a bed 69a). The process of such a plant is sometimes referred to as an RSVE process, where an RSV recycle stream (stream 22b) is typically blended with a GSP reflux (stream 50) prior to entering the fractionator 60. RSVE processes are typically limited to about 97% ethane recovery, whereas, GSP processes are typically limited to around 90 to 94% ethane recovery. In ethane rejection mode, without the added packed bed in the fractionator 60, the process of the plant in FIG. 6 may be limited to 97% propane recovery.

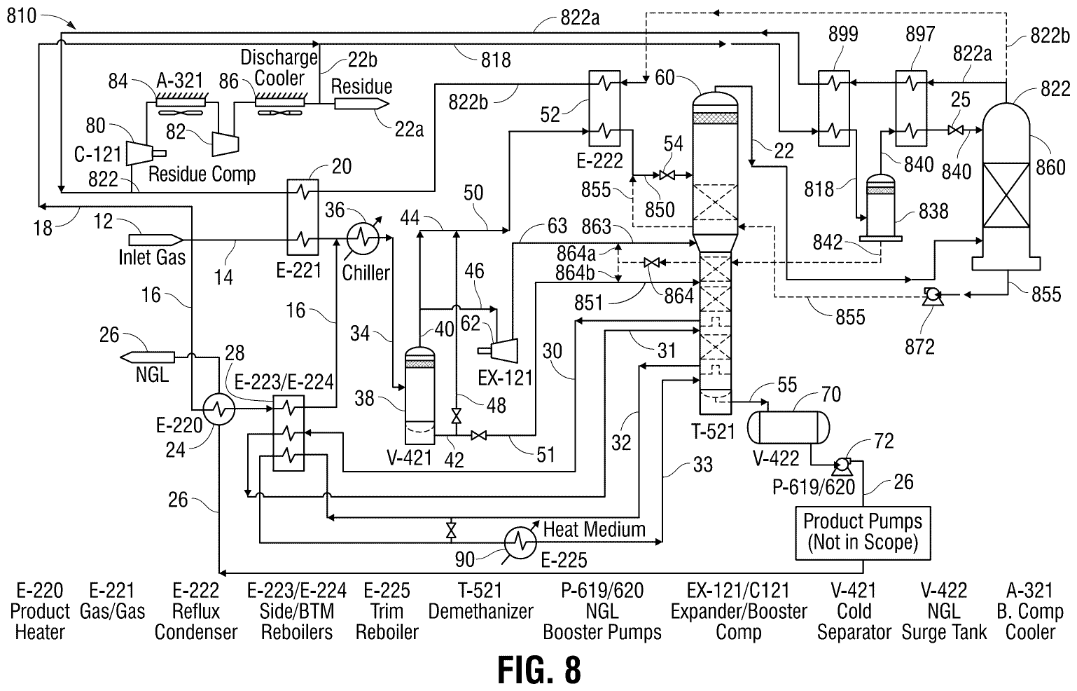

The addition of the second fractionator 660 provides an additional fractionator bed 669 in a separate tower, creating added capacity to the plant 610 while minimizing the need for other equipment (e.g., fractionator 60) to be capable of handling a higher input flow (i.e., minimizing bottlenecks in the plant). The addition of the second fractionator 660 to form plant 610 also provides for a leaner recycle reflux stream 619, which is leaner than the stream 50. FIG. 9 is a graph of exemplary recoveries for a GSP system and an ER-C system.

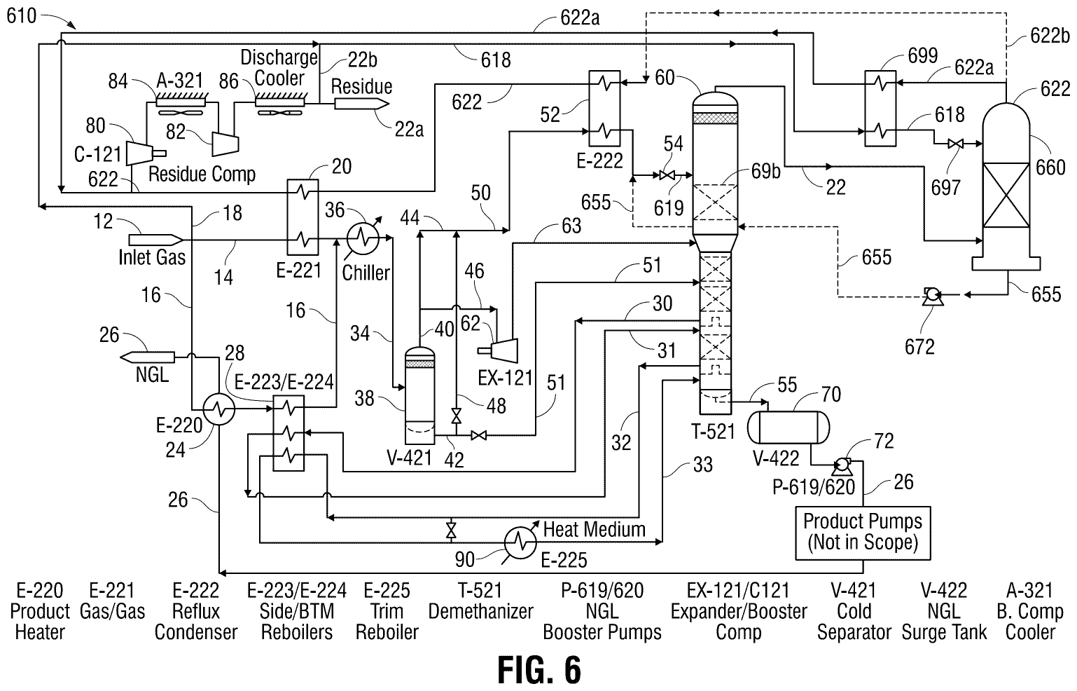

With reference to FIG. 7, plant 710 is similar to the plants shown and described in reference to FIGS. 2-6, with like reference numerals designating like parts. Plant 710 is, in particular, similar to plant 210 of FIG. 2, with the addition of reflux drum 738 (e.g., a second separator), expansion valve 764, and additional piping to provide for the flow of stream to and from the reflux drum 738 and expansion valve 764. In some embodiments, the reflux drum 738 is a stainless steel vessel.

In plant 710, after the third fluid stream 18 passes through the first heat exchanger 20, the third fluid stream 18 is directed through the reflux drum 738. The reflux drum 738 separates the third fluid stream 18 into a top vapor stream 740 and bottom liquid stream 742. The top vapor stream 740 is directed through the fourth heat exchanger 52 to exchange heat with stream 50 and residue gas stream 22. The top vapor stream 740 is directed from the fourth heat exchanger 52, through expansion valve 25 and then into the fractionator 60 at a location above input of the reflux stream 50.

In the embodiment shown in FIG. 7, third fluid stream 18 is entirely inlet fluid gas, and the contents of top vapor stream 740 and bottom liquid stream 742 is determined by what separates at the temperature of the reflux drum 738. For example, if the temperature of the reflux drum 738 is similar to that of the separator 38, then the compositions of top vapor stream 740 and bottom liquid stream 742 will be similar to vapor stream 40 and condensed liquid stream 42. If the temperature of the reflux drum 738 is warmer in comparison to separator 38, then the top vapor stream 740 will be richer with heavier hydrocarbons in it in comparison to vapor stream 40. If the temperature of the reflux drum 738 is cooler in comparison to separator 38, then the top vapor stream 740 will be leaner with of heavier hydrocarbons being in the bottom liquid stream 742 in it in comparison to vapor stream 40. In one example, with an inlet fluid gas 12 containing 80.2 mole % methane, 10.9 mole % ethane, 4.2 mole % propane, and 1.6 butane % mole, the top vapor stream 740 may contain 84.6 mole % methane, 9.9 mole % ethane, 2.8 mole % propane, and a small quantity of heavies. The performance of the reflux drum 738 temperature depends at least in part on the exchangers 20 and 52 and on the mode of operation (e.g., rejection/recovery).

In some embodiments, the reflux drum 738 operates at a relatively high pressure (e.g., similar to separator 38) to separate heavy condensed liquid into the bottom liquid stream 742. Depending on the temperature of the reflux drum 738, the bottom liquid stream 728 may be blended with the cold separated liquids from the separator 38 (i.e., stream 51) or with the stream from the turboexpander 62 discharge (i.e., stream 63). Bottom liquid stream 742 is directed from reflux drum 738 and through valve 764. Valve 764 can direct bottom liquid stream 742 along one or both of the flow paths indicated as bottom liquid streams 741 and 743. Bottom liquid stream 741 is directed to be combined with reflux stream 63, forming combined reflux stream 763 that is directed into the fractionator 60 below reflux stream 50. Bottom liquid stream 743 is directed to be combined with reflux stream 51, forming combined reflux stream 751 that is directed into the fractionator 60 below reflux stream 50 and reflux stream 763. In some embodiments, valve 764 directs all of stream 742 as stream 741. In some embodiments, valve 764 directs all of stream 742 as stream 743. In some embodiments, valve 764 directs a first portion of stream 742 as stream 741 and a second portion of stream 742 as stream 743.

Use of the reflux drum 738 provides for more cold energy in the downstream brazed exchanger (i.e., fourth heat exchanger 52) to condense the cold gas stream (i.e., residue gas stream 22) and to provide a relatively purer reflux to the top of the fractionator 60 (i.e., reflux stream 740). The concepts of FIG. 7 of including a reflux drum can be applied to the process scenarios of either FIG. 2 or 3, optionally in combination with the concepts of either of FIGS. 4 and 5.

By leaning out the affluent stream, top vapor stream 740, and initially separating heavy condensed hydrocarbons, less energy is needed in heat exchanger 52 to cool the stream 740. Thus, more energy (or temperature) is available in stream 22 for thermal exchange in heat exchanger 20. Consequently, with more energy in stream 22 for use at heat exchanger 20, less chilling is required using chiller 36 such that a lower overall HP is required for the plant 710.

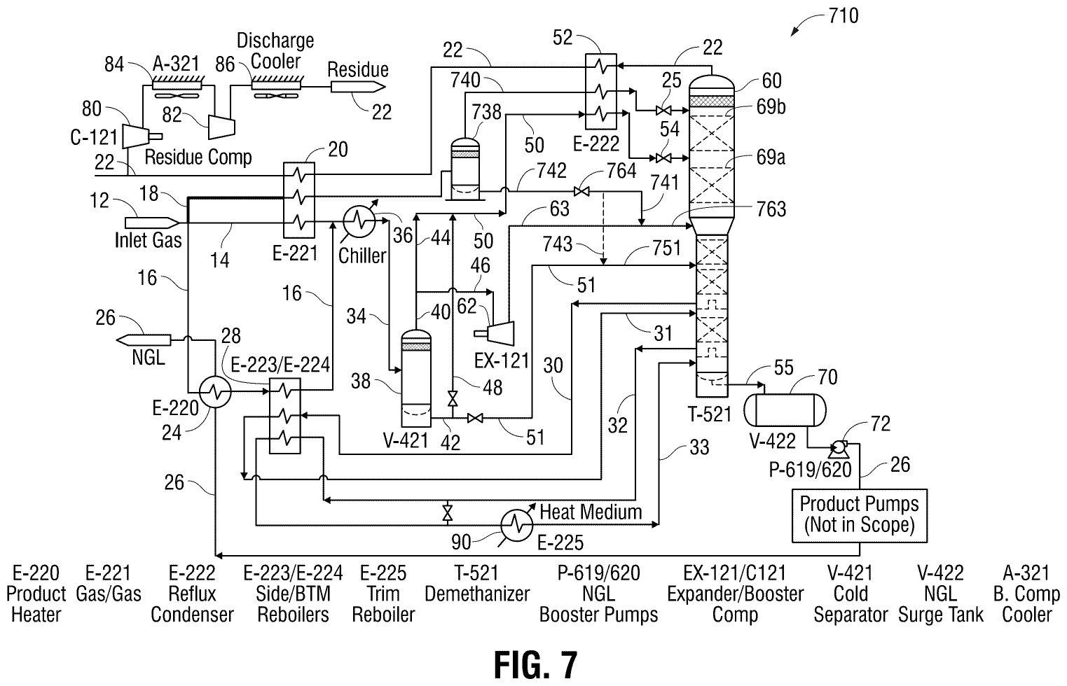

With reference to FIG. 8, plant 810 is similar to the plants shown and described in reference to FIGS. 2-7, with like reference numerals designating like parts. Plant 810 includes reflux drum 838, fifth heat exchanger 899, sixth heat exchanger 897, and second fractionator 860. The embodiment of FIG. 8, in some respects, is a combination of the elements of FIG. 6 with elements of FIG. 7.

In the process of FIG. 8, the third fluid stream 18 is directed to be combined with the second residue gas stream 22b, forming combined stream 818. Combined stream 818 is directed through fifth heat exchanger 899 and then into the reflux drum 838. The reflux drum 838 (which may be the same or similar to separator 738) separates the combined stream 818 to form a bottom liquid stream 842 and a top vapor stream 840.

The bottom liquid stream 842 is directed to valve 864. Valve 864 can selectively direct the bottom liquid stream 842 along one or both of the flow paths indicated as bottom liquid stream 864a and 864b. Bottom liquid stream 864a is directed to be combined with reflux stream 63, forming combined reflux stream 863 that is directed into the fractionator 60 below reflux stream 50. Bottom liquid stream 864b is directed to be combined with reflux stream 51, forming combined reflux stream 851 that is directed into the fractionator 60 below reflux stream 50 and reflux stream 863. In some embodiments, valve 864 directs all of stream 842 as stream 864a. In some embodiments, valve 864 directs all of stream 842 as stream 864b. In some embodiments, valve 864 directs a first portion of stream 842 as stream 864a and a second portion of stream 842 as stream 864b.

The top vapor stream 840 is directed to the sixth heat exchanger 897, through expansion valve 25 and then into the second fractionator 860. The residue gas stream 22 from the fractionator 60 is also fed into the second fractionator 860 at a point below the top vapor stream 840. Within the second fractionator 860, the top vapor stream 840 and the residue gas stream 22 are fractionated to form a top vapor stream 822 and a bottom liquid stream 855. The bottom liquid stream 855 is pumped, via pump 872, to be combined with reflux stream 50, forming reflux stream 850, which is fed into the fractionator 60 after passing through valve 54. The top vapor stream 822 is split into first vapor stream 822a and second vapor stream 822b. First vapor stream 822a is directed through sixth heat exchanger 897 to exchange heat with stream 840, and is directed through fifth heat exchanger 899 to exchange heat with stream 818. Second vapor stream 822b is directed through fourth heat exchanger 52 to exchange heat with reflux stream 50, and then through first heat exchanger 20 to exchange heat with first fluid stream 14. After passing through the heat exchangers 899, 897, 52, and 20, the vapor streams 822a and 822b are recombined to form stream 822.

### Thermal Exchanges

As explained with reference to FIGS. 2-8, the natural gas streams input into the plants are cooled prior to being input into the primary fractionator of the plant. In heat exchanger 20, streams 14 and 18 are the same or substantially the same temperature and stream 22 is colder than both streams 14 and 18. Thus, passing through heat exchanger 20 cools both streams 14 and 18 and heats stream 22. In heat exchanger 52, stream 50 is colder than stream 18, and stream 22 is colder than both streams 18 and 50. Passing through heat exchanger 20 cools both streams 18 and 50 and heats stream 22. In heat exchanger 24 stream 26 is colder than stream 16. Passing through heat exchanger 24 cools stream 16 and heats stream 26. In heat exchanger 28 stream 16 is colder than streams 30 and 32. Passing through heat exchanger 28 cools stream 16 and heats streams 30 and 32. In rejection mode, heat exchanger 90 is activated, heat exchanger 24 is deactivated, and stream 32 bypasses heat exchanger 28 via valve 91 to pass through exchanger 90, warming stream 32 to form stream 33. In some embodiments, phase changes occur in the systems disclosed herein at the exchangers (e.g., at elements 16, 34, 50, 63, 42, and 64). In some embodiments, streams 30 and 31 are liquid draws that become partially vaporized (e.g., via side re-boiling/re-boiling). Referring to FIG. 2, stream 18, between exchangers 20 and 52 may be both liquid and gas phase. The separator (e.g., element 738) can extract heavy hydrocarbons relatively early in the process further cooling in an exchanger (e.g., element 52), which may help avoid freezing of hydrocarbons in the stream.

As evident from the above disclosure, embodiments of the process disclosed herein include bypassing additional inlet gas that would typically pass through the plant by directing the portion of the inlet gas around certain separation stages in the plat. While the bypassing may cause separation and recovery of the plant to reduce slightly, the additional processing capacity (e.g., 10-20% increase or more) can increase revenue sufficiently to justify the bypass without creating bottlenecks in other parts of the plant. That is, recovery is at least partially traded for added capacity.

Any one or more of the process and system elements of any of FIGS. 2-8 can be combined. The enumerated concepts describe, and include within their descriptions, methods, processes, techniques, configurations, systems, apparatus, constructions, assemblies, subsystems and subprocesses and the like. This list should not be considered limiting, however, as, for example, the elements or features, in respect to system or configuration, may be combined with each of the other elements associated with other systems and configurations. The same applies to methods and various, exemplary steps.

The exemplary applications described herein include modifications to an NGL processing plant, and more particularly, techniques favoring the primary recovery of certain targeted hydrocarbons (e.g., from a demethanizer). The described methods and techniques, and system configurations and more detailed variations thereof are not limiting of the concepts. The concepts described herein contemplate, for example, implementation within other NGL processing systems and other recovery techniques for ethane, propane, butane, and/or other hydrocarbons to varying degrees.

### Modifications to Fractionation

Some embodiments of the present disclosure relate, generally, to hydrocarbon processing, and, more directly, to a system, apparatus, and method for the separation of fluids (gas or liquid) containing hydrocarbons. More specifically, embodiments of the disclosed system, apparatus, and method are particularly relevant to separation and recovery techniques in natural gas liquid/liquid petroleum gas (NGL/LPG) processing systems. In that respect, systems, apparatus, and methods disclosed herein are particularly suited or applicable to the separation of ethane, propane, and/or heavier hydrocarbon from such fluid streams.

Some embodiments of the present disclosure include a process for separating a natural gas stream. The process includes directing a first portion of an input stream into a first fractionator. The input stream includes natural gas. The process includes fractionating the input stream within the first fractionator. Fractionating the input stream within the first fractionator forms a product stream and a first vapor effluent stream. The process includes directing the first vapor effluent stream into a second fractionator. The process includes fractionating the first vapor effluent stream within the second fractionator. Fractionating the first vapor effluent stream within the second fractionator forms a first liquid stream and a second vapor effluent stream. The process includes directing the first liquid stream into the first fractionator and fractionating the first liquid stream within the first fractionator while fractionating the input stream within the first fractionator.

Some embodiments of the present disclosure include a system for separating a natural gas stream. The system includes a natural gas inlet and a first fractionator including one or more inlets. At least one of the inlets of the first fractionator is in fluid communication with the natural gas inlet. The first fractionator includes a first vapor effluent outlet and a first liquid product outlet. The system includes a second fractionator. The second fractionator includes one or more inlets in fluid communication with the first vapor effluent outlet. The second fractionator includes a second vapor effluent outlet and a second liquid product outlet. The second liquid product outlet is in fluid communication with at least one of the inlets of the first fractionator.

Some embodiments of the present disclosure include a method for retrofitting a natural gas separation plant that includes a natural gas inlet and a first fractionator including one or more inlets, where at least one of the inlets of the first fractionator is in fluid communication with the natural gas inlet, and where the first fractionator includes a first vapor effluent outlet and a first liquid product outlet. The method includes providing a second fractionator. The second fractionator includes one or more inlets, a second vapor effluent outlet, and a second liquid product outlet. The method includes fluidly coupling at least one inlet of the second fractionator with the first vapor effluent outlet. The method includes fluidly coupling the second liquid product outlet with at least one of the inlets of the first fractionator.

Some embodiments of the present disclosure include a process for the separation of a gas stream. The process includes receiving an effluent gas flow from a first fractionator operating at a first pressure. The process includes splitting the effluent gas flow into a first stream and a second stream. The process includes passing the first stream through a heat exchanger thereby causing a phase change of at least a portion of the first stream from a gaseous state to a liquid state. The process includes inserting the first stream into an upper portion of a second fractionator operating at a second pressure. The second pressure is lower than the first pressure. The process includes inserting the second stream into a lower portion of the second fractionator. The process includes diverting liquids from a lower portion of the second fractionator to the first fractionator.

Some embodiments of the present disclosure include a process for the separation of a gas stream. The process includes directing an effluent gas flow from a first fractionator, and subjecting a portion of the effluent gas flow to a heat exchange. The process includes directing a stream containing at least a portion of said effluent gas flow into a second fractionator. The process includes directing a stream containing liquids from the second fractionator to the first fractionator.

Some embodiments of the present disclosure include a process for the separation of a gas stream. The process includes drawing a stream containing liquids from a secondary fractionator to a main fractionator. The process includes splitting effluent gas flow from the secondary fractionator into a first stream and a second stream. The process includes compressing the second stream prior to directing said compressed stream to the secondary fractionator.

Some embodiments of the present disclosure include a system for processing a natural gas stream for hydrocarbon recovery. The system includes a main fractionator, a secondary fractionator, and an effluent flow line directed as an outlet of the main fractionator. The effluent line is divided into a first flow line and a second flow line. A first heat exchanger is in fluid communication with the first flow line and intermediate the main and secondary fractionators such that a first stream exiting said heat exchanger discharges into an upper portion of the secondary fractionator. The second flow line communicates a second stream from the effluent flow into a lower portion of the secondary fractionator. A lower portion of the second fractionator is disposed in fluid communication with the first fractionator such that a stream containing fluids is communicated from the lower portion to the main fractionator.

Some embodiments of the present disclosure include a system for processing a natural gas stream for hydrocarbon recovery. The system includes a main fractionator, a secondary fractionator, and a first effluent flow line directed from the main fractionator. The first effluent flow line is configured to discharge into to a lower portion of the secondary fractionator. A second effluent flow line is directed from an effluent outlet of the secondary fractionator. The second effluent line is divided into a first flow line and a second flow line in fluid communication with an upper portion of the secondary fractionator. A compressor is disposed in the second flow line downstream of said effluent outlet. A first heat exchanger is disposed in fluid communication with the second flow line and intermediate the effluent outlet and an inlet into an upper portion of the secondary fractionator such that a first stream exiting the first heat exchanger discharges into an upper portion of the secondary fractionator via said inlet. The second flow line communicates a second stream from the effluent outlet into a lower portion of the secondary fractionator. A lower portion of the secondary fractionator is disposed in fluid communication with the main fractionator, such that a stream containing fluids is communicated from the lower portion of the secondary fractionator to the main fractionator.

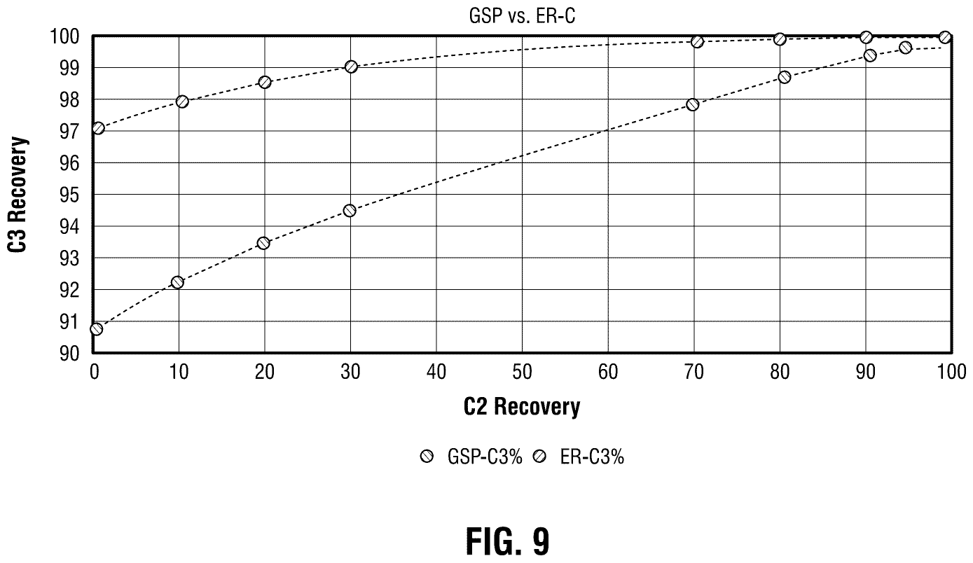

FIGS. 10-14 illustrate exemplary and localized systems, methods and configurations of natural gas processing and recovery, as incorporated within the system and process of FIG. 1A, or as alternatives to the system and process of FIG. 1A. FIG. 10-14 depict embodiments of a hydrocarbon processing plant, plants 10a-10e, that are similar to plant 110a described in respect to FIG. 1A, but modified. Plants 10a-10e and plant 110a are different, as plants 10a-10e include and implements subsystems, subprocesses, techniques, and configurations in accordance with the present disclosure. The subsystems or configurations shown in FIGS. 10-14 can, in some embodiments, be incorporated into an existing system (such as plant 110a) via retrofitting the existing system.

In some embodiments disclosed herein, the relevant system or process is configured or operated to produce an NGL product stream having higher propane recovery and essentially free of ethane (ethane rejection mode). For example, the gas effluent from the fractionator 60 in FIG. 1A, which forms the residue gas stream 22, contains propane not recovered into the liquid product 55 of the fractionator. At least a portion of the propane within the gas effluent from the fractionator 60 can be recovered by passing the gas effluent through a second fractionator (as shown in FIGS. 10-14). In further embodiments disclosed herein, systems and processes are configured and/or operated so as to reduce the occurrence of CO₂ freeze and increase ethane recovery.

In some embodiments according to the present disclosure, the systems or processes are configured and operated to include retrieval of at least a portion of an existing effluent gas from a first fractionator (e.g., demethanizer or deethanizer tower) at a given pressure, of a natural gas plant, and providing the effluent gas into a bottom of a second fractionator tower operating at lower pressure than the first fractionator. The remaining effluent gas, may be passed through a heat exchanger and chilled to produce liquid that is fed to the top of the second fractionator tower. The effluent gas from the second fractionator forms the residue or final gas of the plant. Thus, in some applications, such as a GSP flow-scheme, a top fed, GSP reflux, can be diverted from the first fractionator to a bottom of the second fractionator at a lower pressure. The second fractionator contains liquids in a bottom that are suitable for reflux in the first fractionator. The second fractionator disclosed herein can operate at a lower pressure than the first fractionator. For example, in one embodiment the first fractionator (also referred to as the main fractionator) can operate at a pressure of from 200 psig to 300 psig or higher, and the second fractionator can operate at a pressure that is from 7 to 15 psi lower than the pressure of the first fractionator (e.g., the minimum pressure drop for the process).

FIG. 10 depicts natural gas plant 10a. Natural gas plant 10a is similar to plant 110a shown in FIG. 1A, but includes subsystem 100 incorporated therein to facilitate recovery of certain hydrocarbon components from natural gas, in accordance with the present disclosure. The techniques and system configuration illustrated in FIG. 10 may be implemented for the recovery of hydrocarbons such as ethane, propane, butane, or heavier hydrocarbons.

In the embodiment of FIG. 10, effluent gas stream 102 (which may be the same as stream 22 of FIG. 1A) is withdrawn from the top end of the fractionator 60. The effluent gas stream 102 is then split into a first effluent gas stream 104 and a second effluent gas stream 106.

Combined stream 50, as previously described with reference to FIG. 1A, exits the fourth heat exchanger 52 and passes through a fifth heat exchanger 112. Within the heat exchanger 112, the combined steam 50 is in thermal communication with the second effluent gas stream 106.

After exiting the heat exchanger 112, the combined stream 50 is combined with the first effluent gas stream 104 to form combined stream 113. The combined stream 113 is the fed to a bottom section of a second fractionator 110. In some embodiments, a diameter of the second fractionator is, generally, similar to that of the first fractionator. The second fractionator 110 can include a mass transfer device, such as packing and/or trays. Preferably, the effluent split is about 30% to about 80% flow to fractionator 110, more preferably, about 40%-80%, and normally, about 60%. That is, about 60% of effluent gas stream 102 forms first effluent gas stream 104 to be input into the second fractionator 110 as the bottom, and about 40% of effluent gas stream 102 forms second effluent gas stream 106 that is chilled and becomes a reflux to a top of the second fractionator 110.

The second effluent gas stream 106 is directed through the heat exchanger 112 (e.g., a brazed aluminum heat exchanger) where the second effluent gas stream 106 exchanges heat with the combined stream 50, such that the hotter second effluent gas stream 106 is cooled and the colder combined stream 50 is heated. From the heat exchanger 112, the second effluent gas stream 106 flows into a top section of the second fractionator 110. Typically, the second effluent gas stream 106 is at a temperature of about −90 to −145° F., depending on the temperature of the combined stream 50. Generally, the colder a reflux stream is, the higher recovery achieved with the product steam. Thus, the method preferably includes chilling of second effluent gas stream 106, which is the leanest stream in the plant 10a, to form a reflux to the top of the second fractionator 110 and crossing the second effluent gas stream 106 against the warmer portion of the first effluent gas stream 104 (as mixed with the combined stream 50) entering the bottom of the second new tower. The combination of the leanness of reflux formed by second effluent gas stream 106 and the temperatures and the mass transfer that is exchanged facilitates extraction of additional propane.

In some embodiments, the second fractionator 110 operates at a lower pressure than the pressure at which the fractionator 60 operates. Liquid within the second fractionator 110 is collected at the bottom end of the second fractionator 110, forming a liquid stream 114 that is withdrawn from the second fractionator 112 via pump 116. The pump 116 pumps the liquid stream 114 into the fractionator 60 as a reflux stream 67. The effluent (residue gas stream 22) is drawn from the top of the second fractionator 110.

Thus, relative to the plant 110a of FIG. 1A, subsystem 100 includes an additional fractionator (section fractionator 110), an additional heat exchanger (heat exchanger 112), an additional pump (pump 116), and additional piping to connect these components. The subsystem 100 can be retrofitted into an existing system (e.g., plant 210), such as a GSP system. The subsystem 100 can also be added to cryogenic designs of other plant designs, such as EG Score, Recycle Split Vapor (“RSV”), legacy turboexpander plants without GSP reflux, or other designs. In some embodiments, the system 10a, with subsystem 100, the second fractionator 110 operates at pressure that is about 7 to 15 psi below the pressure at which the first fractionator 60 operates (which typically operates at about 200 to 350 psig or 200 to 270 psig depending upon how much ethane is targeted for recovery). The pressure at the inlet gas 12 is typically higher, such as from 850 to 1100 psi, until the separator 38. Downstream of the separator 38, JT valves and turboexpanders can be positioned to drop the flow(s) to the operating pressure of the fractionator 60, which can create at least some auto-refrigeration.

The temperature of the reflux of the second fractionator is typically colder than that of the first fractionator due to the ratio of volumes chilling the reflux and the leaner composition of the reflux in comparison to the stream chilling the reflux.

TABLE 2
TYPICAL OPERATING TEMPERATURES

| | Existing Demethanizer | Reflux to New | New Fractionator |
|---|---|---|---|
| | Tower Overheads | (Second) Tower | Tower Overheads |
| Case | (° F.) | (° F.) | (° F.) |
| 1 | −66 | −143 | −93 |
| 2 | −72 | −125 | −105 |
| 3 | −55 | −130 | −95 |
| 4 | −131 | −154 | −152 |

Table 3, below, illustrates differences in typical performance between the system and process discussed in respect to FIG. 1A and that of the embodiment discussed in respect to FIG. 10, when each respective plant is run in a deep rejection mode, prioritizing rejection of C₂ (e.g., 0% ethane recovery or substantially 0% ethane recovery). As can be seen, the embodiment in FIG. 10 offers an improvement of more than 6% more C₃ recovered, nearly 2% more isobutane (“iC₄”) recovered, and more than 1% more normal butane (“nC₄”) recovered when compared to the process used in FIG. 1A. This difference in recovery percentages can result in significant economic advantages provided by the embodiment disclosed in FIG. 10. In addition, the embodiment discussed in FIG. 10 may reduce the probability of freezing attributable to CO₂ while also increasing, incrementally, ethane recovery when the plant is in recovery mode. The percent recoveries disclosed herein are mole percent relative to the amount of the constituent in the inlet gas 12. For example, a 98% recovery of propane would mean that 98% of the propane in the inlet gas 12 is recovered in the liquid product 55.

TABLE 3
CHANGE IN PERFORMANCE IN DEEP REJECTION MODE

| | System of | System of | |
|---|---|---|---|
| | FIG. 1A | FIG. 10 | Change |
| Refrigeration Horsepower | 2,193 | 2,332 | 139 |
| Residue Horsepower | 18,378 | 19,084 | 706 |
| C₂ - % Recovered | 0.576 | 0.6185 | 0.0425 |
| C₃ - % Recovered | 91.6 | 98.31 | 6.71 |
| iC₄ - % Recovered | 98 | 99.93 | 1.93 |
| nC₄ - % Recovered | 98.86 | 99.98 | 1.12 |

Table 4, below, illustrates differences in typical performance of the system or process of FIG. 1A and the system or process discussed in respect to FIG. 10, when the two are operated in a typical rejection mode to achieve about 28% to 30% recovery of C₂. As shown, the embodiment of FIG. 10 offers an improvement of more than 4% more C₃ recovered, nearly 1% more iso butane (“iC₄”) recovered, and more than 0.4% more normal butane (“nC₄”) recovered when compared to the process used in FIG. 1A. This difference in recovery percentages can result in significant economic advantages provided by the embodiment disclosed in FIG. 10.

TABLE 4
CHANGE IN PERFORMANCE IN REJECTION MODE

| | System of | FIG. 2 |
|---|---|---|
| | FIG. 1 | Embodiment |
| Plant flow (MMscfd)* | 230 | 230 |
| % C₂ | 28.42 | 29.99 |
| % C₃ | 94.33 | 98.42 |
| % iC₄ | 98.98 | 99.91 |
| % nC₄ | 99.5 | 99.97 |
| Residue HP | 15,771 | 15,473 |
| Refrigeration HP | 2,915 | 4,115 |
| Total HP | 18,686 | 19,588 |

*MMscfd stand for Million Standard Cubic Feet per Day

Table 5, below, illustrates differences in typical performance between operation of the system and process discussed in respect to FIG. 1A and the embodiment discussed in respect to FIG. 10, when each plant is run in a slope recovery mode that operates at about 50% to 57% recovery of C₂. As shown, the embodiment of FIG. 10 offers an improvement of more than 3% more C₃ recovered, nearly 1% more iso butane (“iC₄”) recovered, and more than 0.5% more normal butane (“nC₄”) recovered. This difference in recovery percentages can result in significant economic advantages provided by the embodiment of FIG. 10.

TABLE 5
CHANGE IN PERFORMANCE IN RECOVERY MODE

| | System of | FIG. 2 |
|---|---|---|
| | FIG. 1 | Embodiment |
| Plant flow (MMscfd) | 230 | 230 |
| % C₂ | 56.21 | 51.89 |
| % C₃ | 95.31 | 98.85 |
| % iC₄ | 98.95 | 99.94 |
| % nC₄ | 99.43 | 99.98 |
| Residue HP | 14,411 | 14,246 |
| Refrigeration HP | 4,170 | 4,303 |
| Total HP | 18,581 | 18,549 |

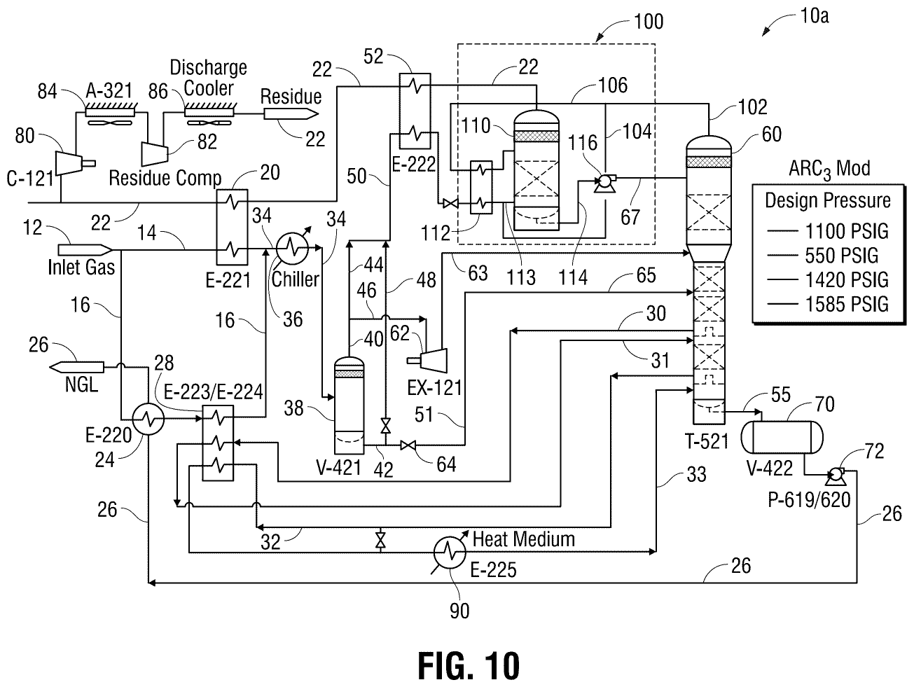

Now turning to FIG. 11, a system and process is provided that illustrates modifications to a natural gas plant 10b to include a subsystem 200 facilitating recovery of certain hydrocarbon components from natural gas, according to the present disclosure. The techniques and system configuration illustrated may be implemented for the recovery of such hydrocarbons as ethane, propane, butane, or heavier hydrocarbons, as will become apparent to one of ordinary skill in the art.

In the embodiment of FIG. 11, effluent gas stream 202 (which may be the same as stream 22 of FIG. 1A) exits the top of fractionator 60. The effluent gas stream 202 is split into a first flow stream 204 and a second flow stream 206. The first flow stream 204 is directed into a sixth heat exchanger 208, wherein the first flow stream 204 is passed in thermal communication with an effluent gas stream 210 from a second fractionator 212. With the heat exchanger 212, the first flow stream 204 is cooled and the effluent gas stream 210 from second fractionator 212 is heated. The first flow stream 204 then directed into a top section of the second fractionator 212. After exiting the heat exchanger 208, the effluent gas stream 210 becomes the residue gas stream 22.

The second flow stream 206 is combined with combined stream 50 (as discussed with respect to FIG. 1A) and the resultant combined stream 207 is directed into the bottom section of second fractionator 212. In other embodiments, the second flow stream 206 flows directly into the second fractionator 212 without mixing with the combined stream 50. In some embodiments, the second flow stream 206 and/or the combined stream 50 flow into a top section of the second fractionator 212. The second fractionator 212 may operate at a lower pressure than the fractionator 60. As such, liquid in the second fractionator 212 is collected at the bottom of the second fractionator 212 to form a liquid stream 214 that is pumped via pump 216 into the fractionator 60 as a reflux stream 219. In some embodiments, the liquid stream 214 is pumped through an expander valve 218 to induce a phase change of at least some of the liquid stream 214—from the liquid phase to the gaseous phase prior to entering the fractionator 60. Thus, system 10b is the same as or substantially similar to system 10a, except that system 10b includes heat exchanger 208 which is positioned differently than heat exchanger 112 and that the liquid stream from the second fractionator passes through an expander valve 218 prior to entering the fractionator 60.

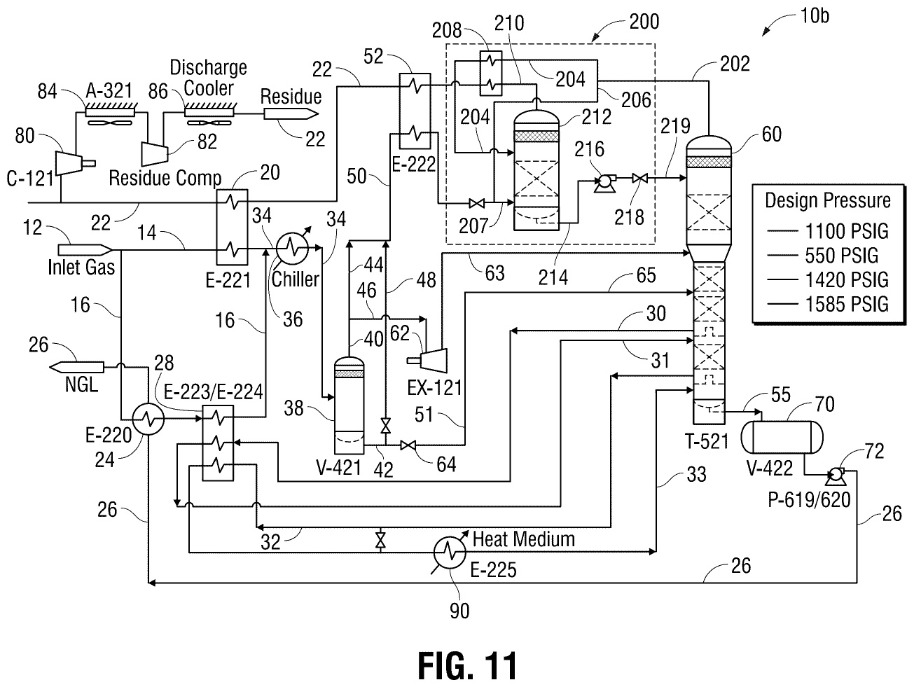

FIG. 12 illustrates yet another system, method, and configuration for the processing of a natural gas liquid/liquid petroleum gas and the separation and recovery of certain hydrocarbons therefrom. As with the exemplary systems, methods, and configurations described above, important aspects are described and illustrated as modifications to the natural gas plant shown in FIG. 1A. System 10c includes a subsystem 300 to facilitate recovery of certain hydrocarbon components from natural gas. The techniques and system configuration illustrated may be implemented for the recovery of such hydrocarbons as ethane, propane, butane, or heavier hydrocarbons, as will become apparent to one of ordinary skill in the art.

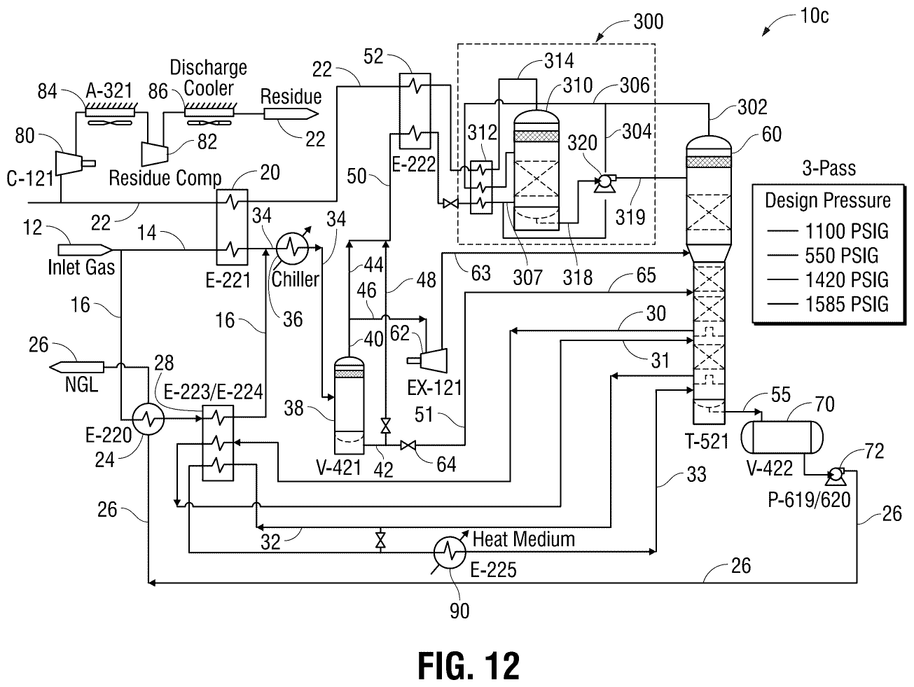

In the embodiment of FIG. 13, effluent gas stream 302 (which may be the same as stream 22 of FIG. 1A) exits the top of fractionator 60 and is then split into a first effluent gas stream 304 and a second effluent gas stream 306. The first effluent gas stream 304 is introduced to combined stream 50 and form combined stream 307, before being passed to a bottom section of the second fractionator 310.

Meanwhile, the second effluent gas stream 306 is directed into a heat exchanger 312, through which effluent gas stream 314 from the top of second fractionator 310 and combined stream 50 also pass through. In this heat exchange, the second effluent gas stream 306 is cooled and then redirected to the top of the second fractionator 310. The effluent gas stream 314 from exiting the top of second fractionator 310 is heated to form the residue gas stream 22. Finally, the combined stream 50 is heated and then combined with the first effluent gas stream 304, prior to being passed to the bottom of the second fractionator 310.

In this embodiment, the second fractionator 310 may operate at a lower pressure than the fractionator 60. The liquid collected at the bottom of the second fractionator 310 is pumped, as liquid stream 318, by pump 320 into the fractionator 60 as reflux stream 319.

FIG. 13 illustrates another exemplary system, method, and configuration according to the present disclosure including exemplary modifications to a natural gas plant. Plant 10d includes subsystem 400 to facilitate recovery of certain hydrocarbon components from natural gas. The techniques and system configuration illustrated may be implemented for the recovery of such hydrocarbons as ethane, propane, butane, or heavier hydrocarbons, as will become apparent to one of ordinary skill in the art.

In the embodiment of FIG. 13, an effluent gas stream 402 (which may be the same as stream 22 of FIG. 1A) exits the top of the fractionator 60 and is then split into first effluent gas stream 404 and second effluent gas stream 406. The first effluent gas stream 404 is combined with combined stream 50 to form combined stream 405 before entering a bottom section of the second fractionator 408.

The second effluent gas stream 406 is directed into a heat exchanger 410 before being fed into the second fractionator 408. Each of the second effluent gas stream 406, the combined stream 50, and the second vapor stream 46 pass through the heat exchanger 410. Within the heat exchanger 410, the second effluent gas stream 406 is cooled prior to flowing into the second fractionator 408, the combined stream 50 is heated prior to combining with the first effluent gas stream 404 and flowing into the bottom of second fractionator 408, and the second vapor stream 46 is heated prior to flowing into the fractionator 60. From the bottom of the second fractionator 408, a liquid stream 412 is pumped back into the fractionator 60 via pump 414 as a reflux stream 419.

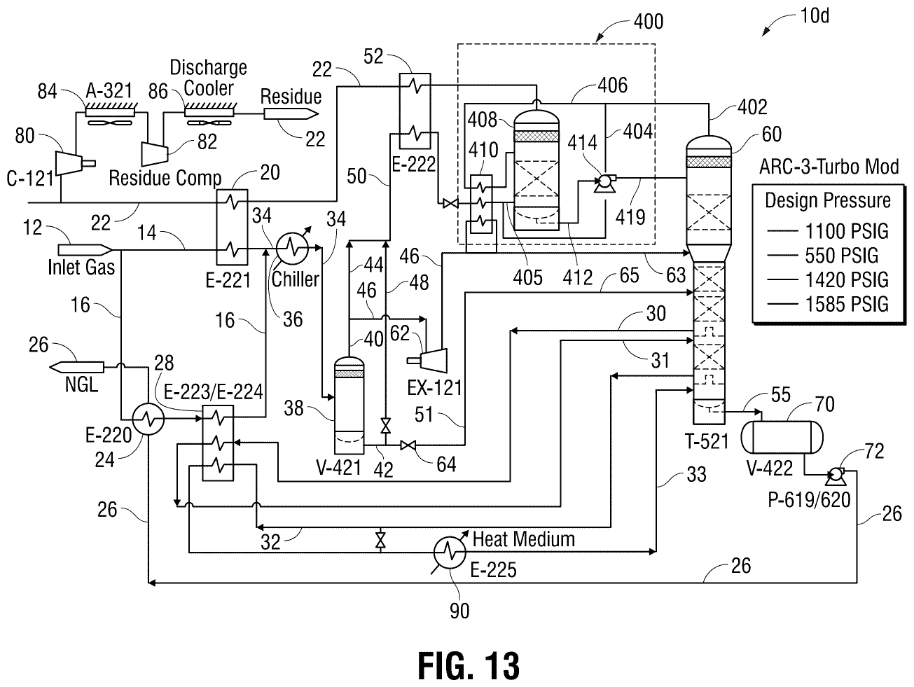

FIG. 14 depicts another exemplary system, method, and configuration according to the present disclosure. FIG. 14 illustrates modifications to a natural gas plant 10e that includes a subsystem 500 to facilitate recovery of certain hydrocarbon components from natural gas, according to the present disclosure. The techniques and system configuration illustrated may be implemented for the recovery of such hydrocarbons as ethane, propane, butane, or heavier hydrocarbons, as will become apparent to one of ordinary skill in the art.

In the embodiment of FIG. 14, an effluent gas stream 502 (which may be the same as stream 22 of FIG. 1A) exits the top of fractionator 60. Unlike the embodiments described in FIGS. 10-13, the effluent gas stream 502 is not split and/or directed to a heat exchanger, but is directed into the fractionator 60. The effluent gas stream 502 is combined with combined stream 50 to form combined stream 503. Combined stream 503 is directed into a bottom of the second fractionator 504. The second fractionator 504 produces a liquid stream 516, which is pumped via pump 518 into the fractionator 60 as a reflux stream 519.

An effluent gas stream 506 is drawn from the second fractionator 504 and split into a first effluent gas stream 508 and a second effluent flow stream 510. The first effluent flow stream 508 forms the residue gas stream 22 after passing through the heat exchanger 52. The second effluent gas stream 510 is compressed (e.g., the pressure of stream 510 can be increased by about 10 psi) via compressor 512. Compression of the second effluent gas stream 510 causes at least some of the stream 510 to phase change from a gaseous state to a liquid state prior to passing into heat exchanger 514. Within heat exchanger 514, the hotter stream 510 exchanges heat (thermal energy) with the colder combined stream 50, prior to the stream 510 moving into a top of the second fractionator 504. In addition, the compression of the stream 510 increases the heat transfer efficiency between the stream 510 and the stream 50 in the heat exchanger 514. The second fractionator 504 may operate at a lower pressure than the fractionator 60. By applying compression, and potentially air cooling before entering the heat exchanger 514, the reflux 510 can be colder and more condensed to enhance propane recovery.

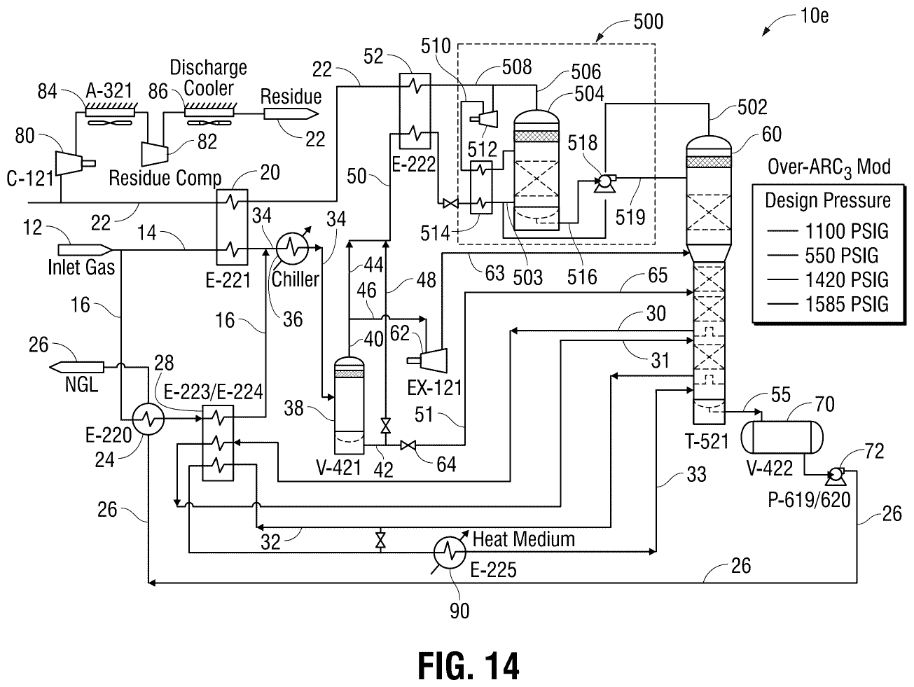

FIG. 15 is a graph showing the propane recovery (y-axis) and ethane recovery (x-axis) for GSP (typical plants being retrofitted) vs. RSV (out of patent Orloff technology) vs. an embodiment of the present disclosure, ARC-3. From left to right on the graph, the C₂ recovery goes from deep rejection (no recovery/0% of ethane) up to a given plant types maximum recovery, generally 90-99%. Both the GSP and ARC-3 processes are limited to somewhere around 90-96% of ethane, whereas, the RSV process can achieve closer to 99% of ethane. The ARC-3 process provides for a steadier recovery and can, at some embodiments, achieve a higher recovery of propane at a given ethane value. The percent recoveries of RSV in FIG. 15 use a high residue gas pressure, which typically results in the highest incremental propane recoveries.

The RSV process is sensitive to residue gas pressure and the amount of recycle flowrate, all of which push recompression horsepower up. FIG. 15 shows an approximately 1200 # residue pressure, which has no impact on the performance of GSP or ARC-3. For the RSV, the amount of slip stream is approx. 15% of plant nameplate (the volume flow used for a reflux, i.e., 15% of available gas). Typically, the higher the flow %, the better the reflux and the better the recovery of propane. However higher flow requires more recompression, such that the recompression HP differential increases from about 20% to about 23 to 25% or higher.

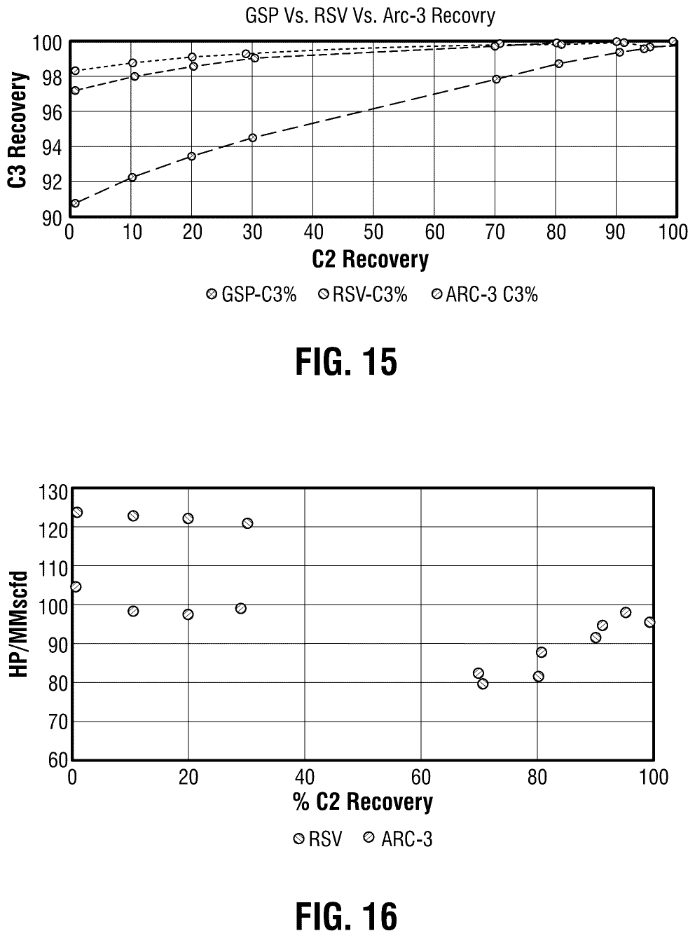

FIG. 16 is a graph of HP/MMscfd (y-axis) and ethane recovery (x-axis). In FIG. 16, the HP, per plant size, show an almost 20% increase in HP requirements in rejection cases to obtain the propane recoveries using RSV. In FIG. 16, the HP per gas plant nameplate shows and almost 20% increase in requirement in rejection for RSV plants to recover high propane, which can be less than, equal to, or slightly greater than the ARC-3 process recoveries.

Many plants use GSP process, while others use RSV. Operational troubles can sometimes cause the RSV recycle to completely shutdown, and the plant reverts to a GSP mode. The ARC-3 embodiment can be used as a retrofit for existing plants, and can facilitate debottlenecking capacity (e.g., a plant at 200 MMscfd flow can be debottlenecked easier at >200 with the ARC-3 retrofit installed).

In some embodiments, the components of the subsystems (e.g., subsystems 100, 200, 300, 400, and 500) can be designed and portions of the equipment utilized to run a plant in an RSV. Such a system can provide incremental ethane recovery in recovery mode (RSV), while requiring the addition of residue compression. Typical GSP recoveries are 90-94%, whereas if a plant is configured with equipment designed to run in RSV mode, the recoveries can be increased to 97-99%. In such a system, the entirety of the gas from the overheads of the main fractionator (e.g., 60) can form directed to a bottom of the second fractionator of the added subsystem. The residue gas can be cooled and become a reflux to the top of the second fractionator.

The enumerated concepts describe, and include within their descriptions, methods, processes, techniques, configurations, systems, apparatus, constructions, assemblies, subsystems and subprocesses and the like. This list should not be considered limiting, however, as, for example, the elements or features, in respect to system or configuration, may be combined with each of the other elements associated with other systems and configurations. The same applies to methods and various, exemplary steps.

The exemplary applications described herein include modifications to an NGL processing plant, and more particularly, techniques favoring the primary recovery of certain targeted hydrocarbons (e.g., from a demethanizer). The described methods and techniques, and system configurations and more detailed variations thereof are not limiting of the concepts. The concepts described herein contemplate, for example, implementation within other NGL processing systems and other recovery techniques for ethane, propane, butane, and/or other hydrocarbons to varying degrees.

The foregoing has been presented for purposes of illustration and description. These descriptions are not intended to limit the disclosure or aspects of the disclosure to the specific plants, systems, apparatus, methods, configurations, and processes disclosed. Various aspects of the disclosure are intended for applications other than the systems or the specific constitution and gas flows referred to above. As noted above, certain of the subprocesses and subsystems may, for example, be readily inserted and substituted in other, similar plant systems and processes. In other words, certain of the processing techniques and methods, and equipment configurations and designs described may also be incorporated into or with other hydrocarbon processing systems and processes. The disclosed systems and methods may also incorporate different components in alternate designs according to the present description. These and other variations of the disclosure will become apparent to one generally skilled in the relevant art provided with the present disclosure. Consequently, variations and modifications commensurate with the above teachings, and the skill and knowledge of the relevant art, are within the scope of the present disclosure. The embodiments described and illustrated herein are further intended to explain best or preferred modes for practicing the disclosure, and to enable others skilled in the art to utilize the disclosure and other embodiments and with various modifications required by the particular applications or uses of the present disclosure.

While specific embodiments and equipment are shown and described herein, one skilled in the art would understand that the methods, systems, and apparatus disclosed herein are not limited to these particular embodiments described. As one of ordinary skill in the art will readily appreciate from the disclosure, systems, processes, machines, configurations, constructions, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

1. A process for separating a natural gas stream, the process comprising:
receiving a natural gas stream;
cooling and separating a first portion the natural gas stream to form a plurality of reflux streams;
directing the plurality of reflux streams into a first fractionator;
cooling a second portion of the natural gas stream and forming an additional reflux stream;
directing the additional reflux stream into the first fractionator;
operating the first fractionator to fractionate the plurality of reflux streams and the additional reflux stream, wherein the operating of the first fractionator forms a first liquid product stream and a first vapor effluent stream; and
withdrawing the first liquid product stream and the first vapor effluent stream from the first fractionator.

2. (canceled)

3. (canceled)

4. The process of claim 1, wherein cooling the first and second portions of the natural gas stream comprises heat exchanging at least a portion of the first and second portions with the first vapor effluent stream.

5-17. (canceled)

18. The process of claim 2, wherein the plurality of reflux streams comprise a first reflux stream and a second reflux stream, wherein first reflux stream is directed into the first fractionator at a location above the second reflux stream.

19. The process of claim 18, wherein the additional reflux stream is directed into the first fractionator at a location above the first and second reflux streams.

20. The process of claim 19, wherein the plurality of reflux streams comprise a third reflux stream that is directed into the first fractionator.

21. The process of claim 20, wherein the additional reflux stream is directed into the first fractionator at a location above the third reflux stream.

22. The process of claim 20, wherein the additional reflux stream is directed into the first fractionator at a location below the third reflux stream.

23. The process of claim 20, wherein the additional reflux stream is combined with the third reflux stream as a combined steam that is directed into the first fractionator at a location above the first and second reflux streams.

24. The process of claim 1, wherein forming the additional reflux stream comprises combining the second portion of the natural gas stream with at least a portion of the first vapor effluent stream.

25. (canceled)

26. (canceled)

27. The process of claim 2, wherein the plurality of reflux streams comprise a first reflux stream and a second reflux stream, wherein forming the additional reflux stream comprises:
directing the second portion of the natural gas stream into a second fractionator;
operating the second fractionator to fractionate the second portion, forming a second liquid product stream and a second vapor effluent stream;
withdrawing the second liquid product stream and the second vapor effluent stream from the second fractionator, wherein the additional reflux stream comprises at least a portion of the second liquid product stream.

28. The process of claim 27, further comprising:
directing the first vapor effluent stream into the second fractionator, wherein the operating of the second fractionator fractionates the second portion of the natural gas stream and the first vapor effluent stream to form the second liquid product stream and the second vapor effluent stream.

29. The process of claim 28, wherein the second portion of the natural gas stream is input into the second fractionator at a location that is above a location where the first vapor effluent stream is input into the second fractionator.

30. The process of claim 27, wherein the additional reflux stream is combined with at least a portion of the first reflux stream and the second reflux stream prior to being directed into the first fractionator.

31. (canceled)

32. The process of claim 27, wherein forming the additional reflux stream comprises combining at least a portion of the second vapor effluent stream with the second portion of the natural gas stream.

33. (canceled)

34. The process of claim 27, further comprising:
separating the second vapor effluent stream into a third vapor effluent stream and a fourth vapor effluent stream;
heat exchanging the third vapor effluent stream with at least a portion of the first portion of the natural gas stream;
heat exchanging the fourth vapor effluent stream with at least a portion of the second portion of the natural gas stream;
recombining the third and fourth vapor effluent streams into a combined vapor effluent stream; and
mixing the combined vapor effluent stream with the second portion of the natural gas stream prior to directing the second input stream into the second fractionator.

35. The process of claim 2, further comprising:
prior to forming the additional reflux stream, directing the second portion of the natural gas stream into a reflux drum;
operating the reflux drum to form a bottom liquid stream and a top vapor stream, and withdrawing the bottom liquid stream and the top vapor stream from the reflux drum, wherein the additional reflux stream comprises at least a portion of the top vapor stream; and
directing the bottom liquid stream into the first fractionator;
wherein the plurality of reflux streams comprise a first reflux stream and a second reflux stream, wherein the operating of the first fractionator fractionates the bottom liquid stream, the first reflux stream, the second reflux stream, and the additional reflux stream to form the liquid product stream and the first vapor effluent stream.

36. The process of claim 35, wherein directing the bottom liquid stream into the first fractionator comprises, prior to directing the first and second reflux streams into the first fractionator, combining at least a portion of the bottom liquid stream with the first reflux stream, the second reflux stream, or combination thereof.

37. The process of claim 2, wherein the plurality of reflux streams comprise a first reflux stream and a second reflux stream, the process further comprising:
directing the second portion of the natural gas stream into a reflux drum and operating the reflux drum to form a bottom liquid stream and a top vapor stream;
directing the bottom liquid stream into the first fractionator;
directing the top vapor stream into a second fractionator;
operating the second fractionator to fractionate at least the top vapor stream to form a second bottom liquid stream and a second top vapor stream in the second fractionator; and
withdrawing the second bottom liquid stream from the second fractionator to form a third reflux stream, wherein the third reflux stream comprises at least a portion of the second bottomer liquid stream.

38. The process of claim 37, further comprising directing the first vapor effluent stream into the second fractionator, wherein operating the second fractionator fractionates the top vapor stream and the first vapor effluent stream to form the second liquid product stream and the second vapor effluent stream.

39. The process of claim 38, further comprising combining at least a portion of the second vapor stream with the second input stream prior to directing the second input stream into the reflux drum.

40. The process of claim 1, wherein the first liquid product stream comprises ethane, propane, butane, or combinations thereof.

41. (canceled)

42. The process of claim 1, wherein the first vapor effluent stream contains propane, methane, or combinations thereof.

43. The process of claim 1, wherein the first fractionator is operated as a demethanizer or a deethanizer.

44. (canceled)

45. (canceled)

46. The process of claim 1, wherein forming the additional reflux stream comprises separating the second portion of the natural gas stream into a vapor stream and a liquid stream, wherein the additional reflux stream comprises at least a portion of the liquid stream.

47-49. (canceled)

50. The process of claim 46, wherein the separating comprises passing at least a portion of the second portion of the natural gas stream through a reflux drum, a second fractionator, or combinations thereof.

51. The process of claim 50, wherein the second portion of the natural gas stream is passed through the reflux drum prior to passing through the second fractionator.

52. A system for separating a natural gas stream, the system comprising:
a first natural gas inlet;
a first separator downstream of and in fluid communication with the first natural gas inlet;
a first fractionator downstream of the first separator, wherein the first fractionator comprises at least two reflux stream inlets in fluid communication with the first separator, wherein a first natural gas flow path is defined from the first natural gas inlet, through the first separator, and into the first fractionator;
a second natural gas inlet upstream of the first fractionator, wherein the first fractionator comprises at least one reflux stream inlet in fluid communication with the second natural gas inlet, wherein a second natural gas flow path is defined from the second natural gas inlet and into the first fractionator;
one or more heat exchangers downstream of the first and second natural gas inlets and upstream of the first fractionator, wherein the one or more heat exchangers are in thermal communication with the first and second natural gas flow paths; and
wherein the first fractionator comprises a first liquid product stream outlet and a first vapor effluent stream outlet.

53-64. (canceled)

65. The system of claim 52, further comprising:
a second fractionator downstream of and in fluid communion with the second input stream, wherein the second fractionator comprises a liquid stream outlet and a vapor stream outlet, wherein the liquid stream outlet is in fluid communication with the at least one reflux stream inlet of the first fractionator; or
a reflux drum downstream of and in fluid communion with the second input stream, wherein the second fractionator comprises a liquid stream outlet and a vapor stream outlet, each in fluid communication with the a reflux stream inlet of the first fractionator; or
a reflux drum downstream of and in fluid communion with the second input stream, wherein the second fractionator comprises a liquid stream outlet and a vapor stream outlet; and a second fractionator downstream of and in fluid communion with the liquid stream outlet of the reflux drum, wherein the second fractionator comprises a liquid stream outlet and a vapor stream outlet, wherein the liquid stream outlet of the second fractionator is in fluid communication with the at least one reflux stream inlet of the first fractionator.

66. The system of claim 52, wherein the heat exchangers comprises at least one three-way heat exchanger positioned to provided thermal exchanger between the first and second natural gas flow paths and the a flow path of a vapor effluent of the first fractionator.

67-74. (canceled)

75. A process for separating a natural gas stream in an existing natural gas processing plant having a first fractionator fractionating separate reflux streams of a first natural gas stream, the process comprising:
cooling a second input stream of natural gas to form a reflux stream comprising at least a portion of the second input stream;
directing the reflux stream into the first fractionator;
operating the first fractionator to fractionate at least the reflux stream and reflux streams of the first natural gas stream, wherein the operating of the first fractionator forms a first liquid product stream and a first vapor effluent stream;
withdrawing the first liquid product stream and the first vapor effluent stream from the first fractionator;
wherein natural gas of the reflux stream flows into the first fractionator along a different flow path than natural gas of the reflux streams of the first natural gas stream.

76-196. (canceled)
